(19) United States
(12) Patent Application Publication
(10) Pub. No.: US 2010/0152646 A1
(43) Pub. Date: Jun. 17, 2010

(54) Intravitreal injection device and method

(76) Inventors: Reshma Girijavallabhan, Denville, NJ (US); Douglas W. Lawrence, Southborough, MA (US); Dana M. Cote, Boxford, MA (US)

Correspondence Address: David W. Highet, VP & Chief IP Counsel;Becton, Dickinson and Company, (Roylance Abrams Berdo & Goodman), 1 Becton Drive, MC 110, Franklin Lakes, NJ, 07417-1880, US

(21) Appl. No.: 12/379,790

(22) Filed: Feb. 27, 2009

Related U.S. Application Data
(60) Provisional application No. 61/064,355, filed on Feb. 29, 2008.

(51) Int. Cl. A61B 17/32; A61M 5/31

(52) U.S. Cl. 604/22

(57) ABSTRACT

A coordinated cutting and spreading mechanism within a syringe dilator sub-assembly is applied to an eye surface during an intravitreal injection to provide an access window free of the conjunctival layer and through which an injection needle can be inserted. The system and method comprises a dilator sub-assembly including both the cutting and spreading mechanism and the intravitreal injection needle, for use with a conventional syringe. The dilator sub-assembly includes a number of projections to secure points of the surface of the conjunctival layer, a cutting member to incise the conjunctival layer, and at least one deflectable projection to move during the intravitreal injection, spreading the incision, and creating a window opening in the conjunctival layer through which the intravitreal injection needle then enters. Upon removal of the device from the injection site, the deflectable projection is released and the window opening in the conjunctival layer is closed.

### CROSS-REFERENCE TO RELATED APPLICATIONS

This application claims the benefit under 35 U.S.C.§119(e) of a U.S. provisional patent application of Reshma C. Girijavallabhan et al., entitled “Intravitreal Injection Device and Method”, Ser. No. 61/064,355, filed on Feb. 29, 2008, the entire content of which is incorporated herein by reference.

### FIELD OF THE INVENTION

The present invention relates generally to ophthalmic injection devices and methods. Specifically, one implementation of the invention relates to an ophthalmic injection device and method that incorporates a mechanism to incise and displace a small portion of the conjunctival layer to create an opening therein prior to and during an intravitreal injection, and to close the opening immediately after the injection.

### BACKGROUND OF THE INVENTION

Intravitreal injection is an effective means of delivering a drug to an interior chamber of the eye. However, a number of concerns exist regarding intravitreal injections generally, and further, when such intravitreal injections are repeated, thereby compounding the possible occurrence of any associated risks.

One concern associated with intravitreal injections is minimizing the tracking of bacteria into the vitreal chamber from the conjunctival layer which covers the sclera, when performing an intravitreal injection. As the conjunctival layer is prone to bacteria, each device touching the bacteria-laden conjunctival layer risks picking up unwanted bacteria. As a result, each device being inserted, or each intravitreal injection being performed, may carry with it bacteria from the conjunctival layer before entering the immunologically deficient vitreal chamber. Such bacteria can then result in endophthalmitis, a serious infection of the eye.

For example, endophthalmitis can occur due to the needle tracking through the heavily bacterial-laden conjunctival layer before entering the immunologically deficient vitreal chamber or by entry through the opening created by the intravitreal injection. In at least one study, the current incidence of endophthalmitis is 1 in 1000 injections. As the frequency of intravitreal drug delivery increases, the cumulative incidence of endophthalmitis can reach 1 in 100.

As noted, bacteria entering the vitreal chamber may increase the risk of developing endophthalmitis. Such entry can occur, for example, by the piercing member that first contacts the bacteria-laden conjunctival layer and then proceeds to travel all the way into the vitreal chamber. Bacteria can be tracked through the opening by the injection needle, or may simply enter the opening during or after injection since the conjunctival layer remains surrounding the opening. Such issues are often associated with vitreous reflux and/or vitreous wicking, wherein the withdrawn needle withdraws vitreous material and provides a vitreous pathway into the vitreal chamber.

One method to address the above risks is the use of a substance such as betadine to treat the intravitreal injection site. The application of betadine is beneficial to a degree in that the injection site is more clearly marked by the colored betadine and is effectively disinfected at the application site. However, the application of betadine can irritate the eye to such an extent that it is left in place only briefly before the intravitreal injection to minimize discomfort.

Therefore, a need exists to provide an intravitreal injection device and method of use that prevents or minimizes contact between the piercing member and the bacteria-laden conjunctival layer, to prevent bacterial tracking and entry into the injection site prior to, during, and after an intravitreal injection.

### SUMMARY OF THE INVENTION

An aspect of one implementation of the present invention is to provide an intravitreal injection device having a cutting and spreading mechanism, wherein the cutting and spreading mechanism is engaged when the intravitreal injection device contacts the surface of the eye to create a temporary opening through the bacteria-laden conjunctival layer through which an intravitreal injection is performed.

Accordingly, an aspect of one implementation of the present invention is to provide an intravitreal injection device which preserves the sterility of the immunologically non-privileged vitreal chamber prior to, during and after an intravitreal injection.

Another aspect of one implementation of the present invention is to provide an intravitreal injection device in which the cutting and spreading mechanism is provided as the substantially sole contact member with the conjunctival layer, and the intravitreal injection needle is prevented from contacting the conjunctival layer.

Another aspect of one implementation of the present invention is to provide an intravitreal injection device in which the cutting and spreading mechanism is used to incise the conjunctival layer and create an opening therein prior to engagement by the needle, such that the needle never contacts the outer layers of the eye when entering the vitreal chamber.

Another aspect of one implementation of the present invention is to provide an intravitreal injection device in which the cutting and spreading mechanism provides a small incision in the conjunctival layer and secures a number of points of the conjunctival layer about the incision while at least one point is gently displaced away from the incision to spread the incision opening and create an access window free of the conjunctival layer and through which the injection needle can be inserted, and from which the injection needle can be retracted.

Another aspect of one implementation of the present invention is to provide an intravitreal injection device in which a sterile needle is provided for the intravitreal injection, and which does not contact the conjunctival layer prior to, during or after the intravitreal injection.

Another aspect of one implementation of the present invention is to provide an injection device in which the cutting and spreading mechanism releases the secured and displaced points of the conjunctival layer upon completion of the intravitreal injection such that the incision of the conjunctival layer is substantially closed.

Another aspect of one implementation of the present invention is to provide an injection device in which the device can be removably coupled with a syringe such that the syringe provides a gripping surface for engaging the device with the injection site such that pressing the gripping surface toward the injection site automatically operates both the cutting and spreading mechanism, and the injection mechanism.

Another aspect of one implementation of the present invention is to provide an injection device in which the automatic operation of both the cutting and spreading mechanism, and the injection mechanism, are arranged to operate in a coordinated manner such that the access window free of the conjunctival layer is created first, and then the injection needle is inserted.

Another aspect of one implementation of the present invention is to provide an injection device in which the cutting and spreading mechanism, and the injection mechanism, can be collectively provided as a hub assembly to be removably secured to a syringe.

Another aspect of one implementation of the present invention is to provide a syringe for receiving the hub assembly including the cutting and spreading mechanism, and the injection mechanism.

These and other aspects are substantially achieved by providing a system and method for an intravitreal injection device incorporating a coordinated cutting and spreading mechanism to provide an access window free of the conjunctival layer and through which an injection needle can be inserted and from which the injection needle can be retracted. The system and method comprises hub assembly for use with a conventional syringe. The hub assembly comprises a substantially cylindrical body having a slidable outer gripping surface. Once engaged with a syringe, and pressed against an injection site, the outer gripping surface remains stationary upon the injection site while the syringe body is slidable toward the injection site within the outer gripping surface. The outer gripping surface has a substantially circular proximal end, from which a number of projections extend to secure points of the surface of the conjunctival layer. A cutting member is provided to gently contact and incise the conjunctival layer. At least one of the projections securing a point of the conjunctival layer is cantilevered to be movable away from the injection site and the incision. To do so, the at least one projection includes an inclined inner surface which is engaged by movement of the syringe toward the injection site such that the projection is gently deflected away from the injection site and the incision, thereby moving the secured point of the conjunctival layer away from the incision and creating a window opening in the conjunctival layer through which the intravitreal injection needle then enters. The syringe is then activated completing the injection. Upon removal of the intravitreal injection device from the injection site, the injection needle will retract out of the clean open widow and the at least one projection that includes the inclined inner surface, is released by movement of the syringe away from the injection site such that the projection is gently moved toward the injection site and the incision, thereby moving the secured point of the conjunctival layer toward the incision and closing the window opening in the conjunctival layer.

Further objectives and advantages, as well as structures and functions of exemplary embodiments, will become more apparent from a consideration of the following description, drawings and examples.

### BRIEF DESCRIPTION OF THE DRAWINGS

These and other objects, advantages and novel features of the invention will become more readily appreciated from the following detailed description when read in conjunction with the accompanying drawings, in which:

FIGS. 1A and 1B are perspective views of an intravitreal injection device comprising a syringe and a dilator sub-assembly configured to perform as a conjunctival dilator according to an embodiment of the present invention;

FIG. 2 is an enlarged sectional view of the dilator sub-assembly configured to perform as a conjunctival dilator prior to engagement according to an embodiment of the present invention;

FIG. 3 is an enlarged sectional view of the distal end of the dilator sub-assembly prior to engagement according to an embodiment of the present invention;

FIG. 4 is an enlarged front view of the distal end of the dilator sub-assembly prior to engagement according to an embodiment of the present invention;

FIG. 5 is an enlarged perspective view of the dilator sub-assembly in relation to the syringe according to an embodiment of the present invention;

FIG. 6 is an enlarged sectional view of the dilator sub-assembly during engagement and showing deflection of a dilating foot according to an embodiment of the present invention;

FIG. 7 is an enlarged sectional view of the dilator sub-assembly during insertion of the intravitreal injection needle of the syringe according to an embodiment of the present invention;

FIG. 8 is an enlarged perspective view of the dilator sub-assembly illustrating a status, or stroke, window according to an embodiment of the present invention;

FIG. 9 is another enlarged sectional view of the dilator sub-assembly during insertion of the intravitreal injection needle according to an embodiment of the present invention;

FIGS. 10A-10O are views of exemplary cutting members according to an embodiment of the present invention;

FIGS. 11A and 11B are views of an exemplary compression spring according to an embodiment of the present invention;

FIGS. 12A-12C are views of an exemplary cam piece according to an embodiment of the present invention;

FIGS. 13A-13C are views of an exemplary dilator sub-assembly according to an embodiment of the present invention;

FIG. 14 is a perspective view of a syringe to which the dilator sub-assembly configured to perform as a conjunctival dilator can be attached according to an embodiment of the present invention;

FIGS. 15A-15D are perspective views illustrating an exemplary assembly of the syringe with the dilator sub-assembly configured to perform as a conjunctival dilator according to an embodiment of the present invention;

FIGS. 16A-16D are perspective views illustrating an exemplary use of the syringe with the dilator sub-assembly configured to perform as a conjunctival dilator according to an embodiment of the present invention; and

FIG. 17 is an illustrative view of an injection site showing exemplary positions, movements and openings created with the dilator sub-assembly configured to perform as a conjunctival dilator according to an embodiment of the present invention.

In the drawing figures, it will be understood that like numerals refer to like structures.

### DETAILED DESCRIPTION OF THE EXEMPLARY EMBODIMENTS

Exemplary embodiments of the present invention provide an ophthalmic injection device and method that incorporates a mechanism to incise and displace a small portion of the conjunctival layer to create an opening or window therein prior to, during and after an intravitreal injection, and to close the opening after the injection needle is removed and the injection completed. Specifically, exemplary embodiments of the present invention are provided to move the bacteria-laden conjunctival layer above the scleral tissue aside such that the needle only penetrates scleral tissue before entering the vitreal chamber, into which it will inject fluids (“intravitreal injection”).

The exemplary embodiments of the present invention incorporate a coordinated cutting and spreading mechanism, or conjunctival dilator, to provide an access window free of the conjunctival layer and through which an injection needle can be inserted and from which the injection needle can be retracted. To do so, the system and method comprises a removable hub assembly including both the cutting and spreading mechanism and the intravitreal injection needle, for use with a conventional syringe.

More specifically, the exemplary embodiments of the present invention describe a conjunctival dilator this is configured to move the bacteria-laden conjunctival layer above the scleral tissue aside such that a needle only penetrates scleral tissue before entering the vitreal chamber into which it will inject fluids, thereby performing an intravitreous injection. The exemplary embodiments of the conjunctival dilator are preferably comprised of four components including a syringe with a hypodermic needle and hub, a compression spring, a cam and dilator sub-assembly. The exemplary conjunctival dilator sub-assembly has three stationary feet, one dilating or movable foot, and a blade. The stationary feet can have dull distal ends to avoid cutting or damaging the conjunctival tissue, and the dilating foot can have a sharper distal end that is able to grip and dilate, or gape, the conjunctival tissue. The hub of the device, into which a needle is assembled, is preferably attached to a standard Luer-Lok™ syringe filled with medication/fluid. The conjunctival dilator can be configured to be compatible with any syringe with a Luer-Lok type distal end, or any number of other syringe confugrations. The syringe barrel can be filled by the user aspirating medication from a vial or is pre-filled with the intravitreous medication from the supplier. The syringe and hub are secured to the cam, which can be slidably captured within the dilator sub-assembly as described in greater detail below.

The exemplary conjunctival dilator is configured to locate the injection site, incise the conjunctiva, dilate the conjunctival incision, and provide visualization of the injection stroke. The four feet of the dilator sub-assembly are arranged such that their tips preferably form a 7 mm diameter circle, but are not limited thereto. This aids the user in achieving the desired 3.5 mm injection distance from the eye's limbus as described in greater detail below. Three of the feet of the dilator sub-assembly are stationary and at least one is moveable, thereby operating as the dilating foot. A cam follower surface is provided to generate the movement of the dilating foot as described in greater detail below. Cam stops extending from the cam, and cam stop lead-in areas of the dilator sub-assembly enable the cam stops to line up when being inserted into the dilator sub-assembly, and provide a ramp to facilitate a snap-fit of the two components.

A blade and blade slot are also provided with the dilator sub-assembly, wherein the blade slot is configured to house the blade or cutting member, and a needle clearance hole is provided also provided with the dilator sub-assembly to allow sufficient room for the needle of the hub to freely move concentrically inside the dilator sub-assembly. A blade stop is also provided with the dilator sub-assembly and is configured to limit the depth that the blade can cut into the conjunctiva and/or sclera.

The cam is also configured anchor a compression spring onto the needle's hub, such that the spring once in position within the dilator sub-assembly provides a constant urging force pushing the cam and hub away from an injection position. The cam is also configured to provide motion to the dilator's dilating foot when advanced toward the injection position against the spring, and the cam stops as exposed and captured within stroke windows in the dilator sub-assembly provide visualization of the injection stroke.

In an exemplary use described in greater detail below, the cam stop is configured to line up with the dilator sub-assembly cam stop lead-in ramp to facilitate a snap-fit into the dilator sub-assembly stroke window. In-use, this feature provides visualization of the injection stroke as the cam moves through the stroke window. the cam further provides a compression spring clearance hole that is configured such that the cam can slide over the compression spring's outside diameter. A back portion of the cam is configured to press-fit the cam and compression spring onto the needle's hub to thereby secure the cam to the hub, and secure one end of the compression spring as well. A front portion of the cam is configured to allow the compression spring to move freely inside the cam, and a cam lip is provided to extend some distance form the front portion. The cam lip pushes on the dilating foot of the dilator sub-assembly or more specifically, pushes on the cam follower surfaces of the dilating foot to provide the dilating foot's movement. A cam follower clearance slot is also provided to allow clearance such that only the cam lip engages with the dilator's cam follower surfaces, and not the cam's outside diameter.

As described in greater detail below, the compression spring provides pressure between the syringe/hub and the dilator sub-assembly. This pressure keeps the dilator's feet anchored in the conjunctiva and enables the dilator sub-assembly and needle/hub sub-assembly to retract back to its initial position after completion of the injection. As noted above, embodiments of the present invention are configured to be used with a conventional syringe. Such a syringe can comprise a syringe barrel having an outside diameter that permits it to slide inside the dilator sub-assembly inside diameter. A hub taper can provide an area to which the compression spring and the cam can be anchored or secured to the needle/hub sub-assembly. The syringe barrel's leading edge in conjunction with the cam lip, push on the cam follower surfaces of the dilating foot to provide the dilating foot's movement. After extension of the injection needle, further pushing on the plunger will inject medication through the needle into the eye.

The dilator sub-assembly comprises a substantially cylindrical body having an outer gripping surface, and an inner bore which can slidably capture a cam piece for movement within a fixed stroke distance, wherein the cam can be secured to a hub and/or syringe, such that the syringe body can also slidably travel within a proximal end of the dilator sub-assembly. In an exemplary embodiment, the dilator sub-assembly comprises a needle and needle hub, the hub comprising a fitting mechanism for engagement with the syringe, and a cam piece which surrounds the needle hub and which is preferably fitted in some manner to the needle hub. Once the hub and cam are captured within the dilator sub-assembly and secured to the syringe, the syringe can be used to press the opposite end of the dilator sub-assembly against an injection site. The dilator sub-assembly remains stationary upon the injection site, while the syringe, needle and needle hub, and cam piece are slidable toward the injection site within the dilator sub-assembly.

The dilator sub-assembly further comprises a substantially circular proximal end, from which a number of projections extend as feet to secure points of the surface of the conjunctival layer at the injection site. In an exemplary embodiment of the present invention, arcuate projections can be provided to have a diameter coaxial with the injection needle. Further, each projection can have a tapered, pointed or rounded end, to facilitate securing the points of contact with the conjunctival layer without cutting or damaging the conjunctival layer.

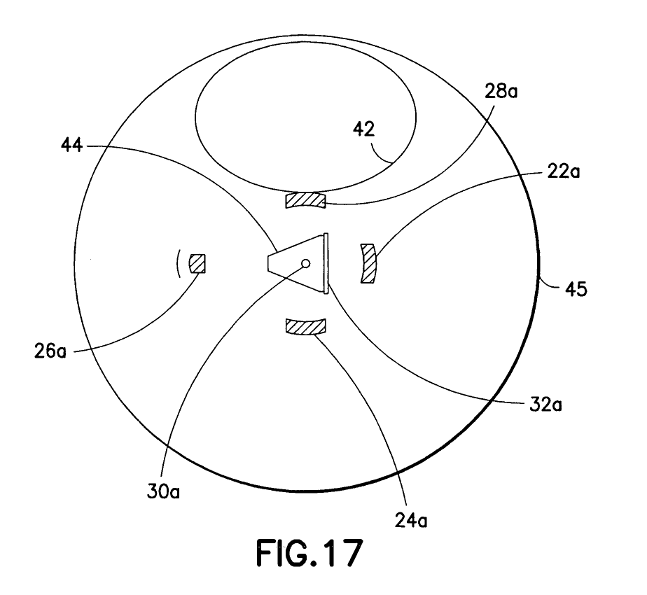

The cutting member of the dilator sub-assembly can be provided within the diameter of the projections and adjacent to the injection needle axis to gently contact and incise the conjunctival layer at the injection site. At least one of the projections securing a point of the conjunctival layer, referred to as the dilating foot, is cantilevered with the dilator sub-assembly to be movable away from the injection site and the incision created by the cutting member. To do so, at least one projection includes an inclined inner surface, or ramp, which is engaged by movement of the cam lip of the cam during movement of the syringe, needle and needle hub and cam piece within the dilator sub-assembly, such that the ramp of the displaceable projection contacts the cam lip of the cam piece and is gently deflected away from the injection site and the incision, thereby displacing the secured point of the conjunctival layer away from the incision and creating a window opening in the conjunctival layer through which the intravitreal injection needle can then enter. That is, as shown in FIG. 17, an opening in the conjunctival layer is provided substantially defined by the width of the incision created by the cutting member on one side and the displaced distance of the deflected projection at the opposite side. The syringe is then activated completing the intravitreal injection. When the syringe body, needle hub and cam piece are relaxed and moved away from the injection site within the dilator sub-assembly, the injection needle will retract out of the clean open widow and the ramp of the displaceable projection is released by the cam lip of the cam piece, and gently returns to the non-deflected position, thereby returning the secured point of the conjunctival layer toward the incision and closing the window opening in the conjunctival layer.

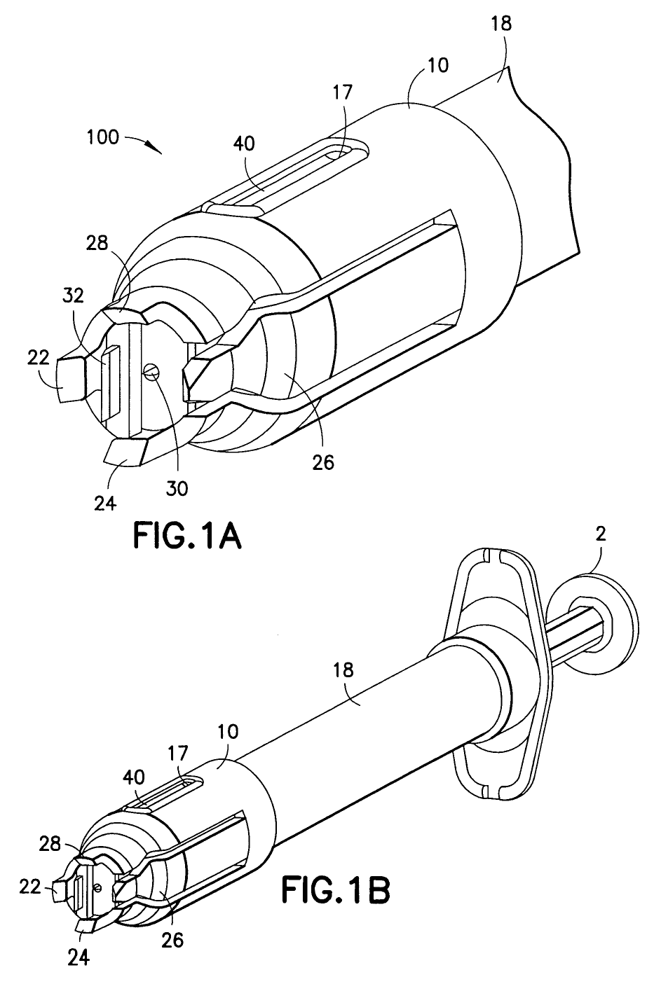

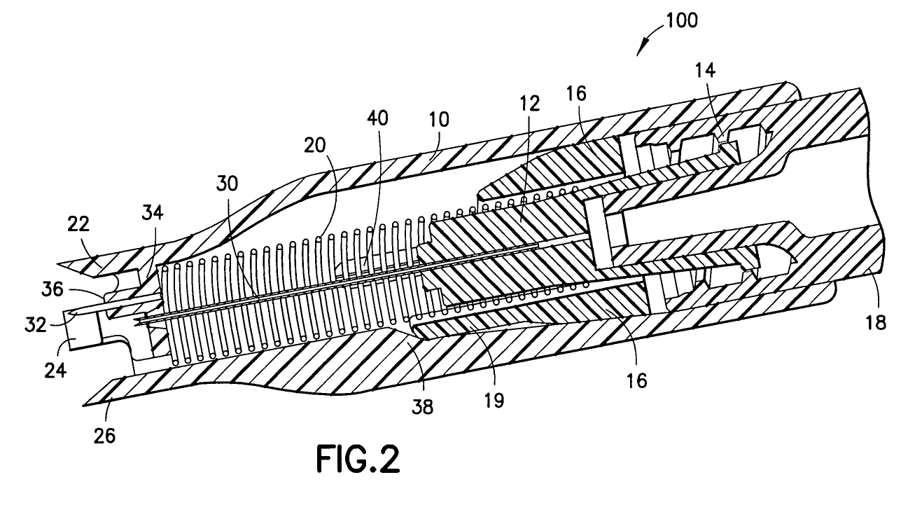

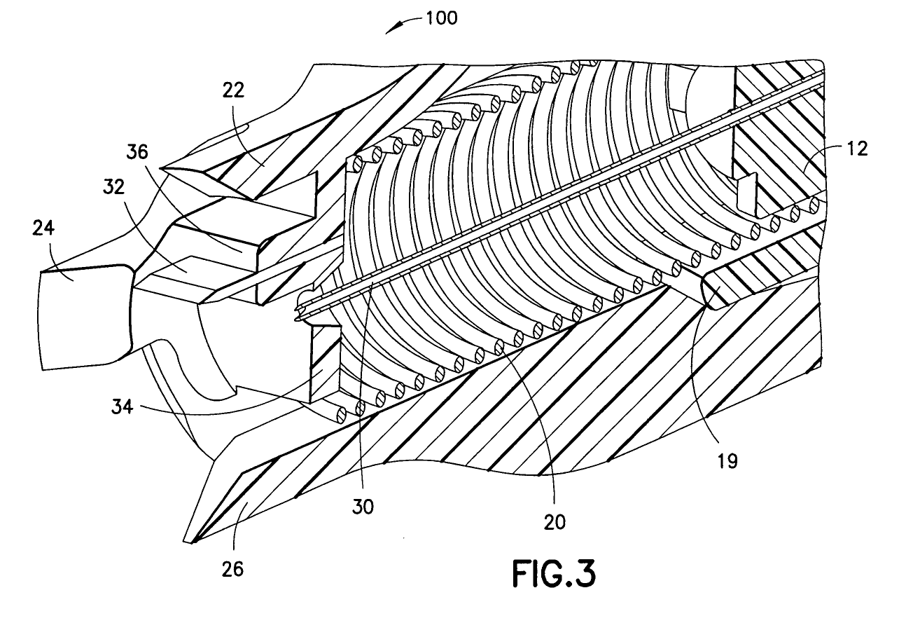

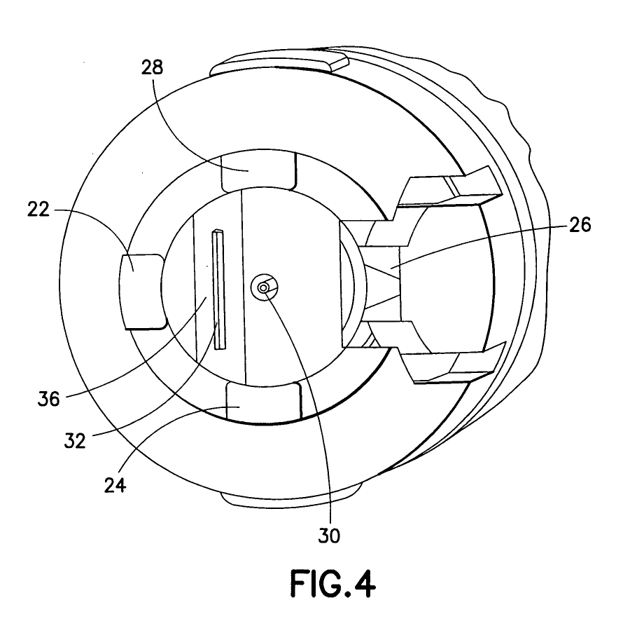

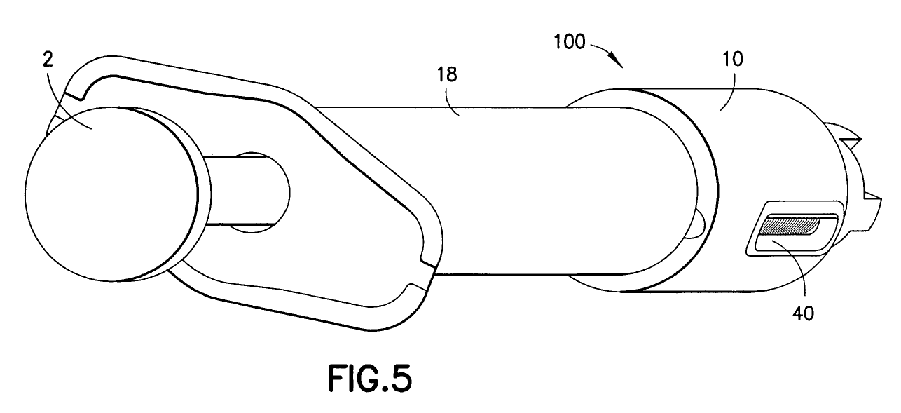

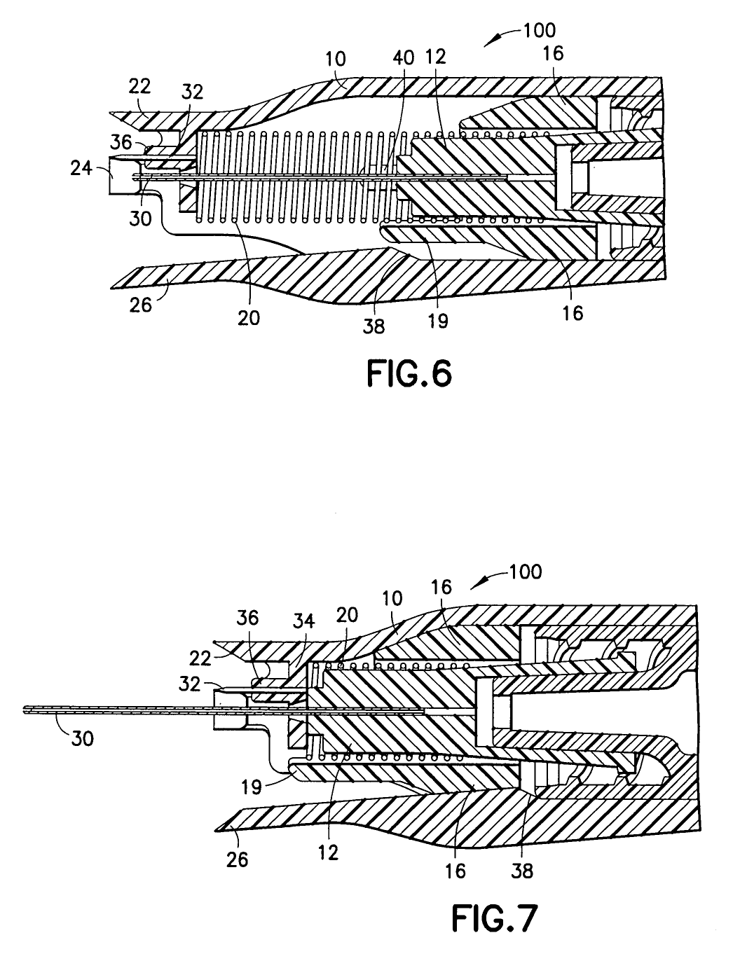

FIGS. 1A and 1B are perspective views of an intravitreal injection device comprising a syringe and dilator sub-assembly configured to perform as a conjunctival dilator according to an embodiment of the present invention. FIG. 2 is an enlarged sectional view of an intravitreal injection device prior to engagement according to an embodiment of the present invention. FIG. 3 and FIG. 4 are enlarged views of the distal end of the intravitreal injection device and FIG. 5 is an enlarged perspective view of the intravitreal injection device in relation to a syringe. FIG. 6 is an enlarged sectional view of the intravitreal injection device during engagement and showing deflection of at least one projection, and FIG. 7 is an enlarged sectional view of the intravitreal injection device during insertion of the intravitreal injection needle. Each position is described in greater detail below.

In FIGS. 1A and 1B, a dilator sub-assembly 100 is shown comprising a substantially cylindrical body having an outer gripping surface, and an inner bore which can slidably capture a cam piece for movement within a fixed stroke distance, wherein the cam piece can be secured to a hub and/or syringe, such that the syringe body 18 can also slidably travel within a proximal end of the dilator sub-assembly. As shown in greater detail in FIG. 2, the dilator sub-assembly 100 comprises a dilator housing 10 which contains a needle hub 12 and fitting mechanism 14, and a cam piece 16 which surrounds the needle hub 12. In an exemplary embodiment of the present invention, the dilator housing 10 comprises a substantially cylindrical member having an open proximal end for allowing insertion of the spring 20, cam piece 16, and needle hub 12 during assembly, and for allowing engagement between the hub 12 and the syringe to thereby allow the slidable movement of the syringe body within the dilator sub-assembly.

The dilator housing 10 further comprises a reduced diameter distal end for contact with an intravitreal injection site. Once engaged with a syringe body 18 and pressed against an injection site, the dilator housing 10 remains stationary upon the injection site while the syringe body 18, needle hub 12 and cam piece 16 are slidable toward the injection site within the dilator housing 10. The slidable movement toward the injection site is opposed by a spring 20, as captured within the dilator housing 10 between the distal end and the needle hub 12. The needle hub 12 is captured within the dilator housing 10 through engagement between the needle hub 12 and the cam piece 16, wherein the cam piece is captured within the dilator housing 10 by the cam stop projections disposed within the stroke windows of the dilator housing 10 as described in greater detail below.

The substantially circular distal end of the dilator housing 10 of the dilator sub-assembly 100 comprises a plurality of projections, or feet, 22, 24, 26 and 28 positioned at approximately 0°, 90°, 180° and 270° about the circular distal end. Although four arcuate projections are shown and/or described in the exemplary embodiment of the present invention, more clearly shown in FIGS. 2 and 3, the present invention is not limited thereto. Any number or shape of projections as desired by the application can be used.

In an exemplary embodiment of the present invention, the projections 22, 24, 26 and 28 function as three stationary feet 22, 24 and 28, and one movable or dilating foot 26, positioned about a cutting member 32 and coaxial with an injection needle 30. The stationary foot 22 opposite to the dilating foot 26 can be considered the primary anchor point. The stationary feet 24 and 28, as with the other feet, preferably have distal ends that are configured to anchor the intravitreal injection device into the tissue of the conjunctival layer, but not to cut or damage the conjunctival tissue. The dilating foot 26 preferably has a pointed distal end to be able to manipulate the conjunctival tissue as described in greater detail below. That is, the end of the dilating foot 26 is configured to secure a point of the conjunctival surface such that when the dilating foot is moved, the conjunctival tissue at the point of contact with the foot is moved in a substantially similar manner with minimal slipping or damage to the tissue.

The projections 22, 24, 26 and 28 extend as separated members from the dilator housing 10 to secure points of the surface of the conjunctival layer, and can be provided to have a diameter, as defined by the arcuate curvature of each, that is coaxial with the injection needle 30, and which surrounds the cutting member. Each projection 22, 24, 26 and 28 can have a tapered, pointed or rounded end, to facilitate securing the points of contact with the conjunctival layer. In the exemplary embodiment of the present invention shown, the projection or foot 22 serves as an anchor point, the projections or feet 24 and 28 serve as additional stationary feet, and the projection 26 serves as the movable or dilating foot. As described in greater detail below, the dilating foot 26 is cut from the dilator sub-assembly in a manner to be cantilevered. Further, the dilating foot 26 comprises a number of additional features upon an inner surface, within the inner bore of the dilator sub-assembly such that the cam lip and cantilever features can be used to displace the dilating foot at precisely the correct movement to provide the access window before injection needle extension from the dilator sub-assembly. Further, the projections 22, 24 and 28 are illustrated having an arcuate end to secure the points of contact with the conjunctival layer, and projection 26 is illustrated as tapering to a sharp point, but each are not limited thereto. Other configurations can be provided as desired by the application.

The cutting member 32 can be provided within the circumference of the projections 22, 24, 26 and 28 to gently contact and incise the conjunctival layer at a point adjacent to the desired injection site and opposite to the dilating foot. In an exemplary embodiment of the present invention, the cutting edge of the cutting member 32 extends to distance substantially equal to the distance of the projections 22, 24, 26 and 28, although the present invention is not limited thereto. The cutting edge of the cutting member 32 can extend to a greater distance or a lesser distance as desired by the application.

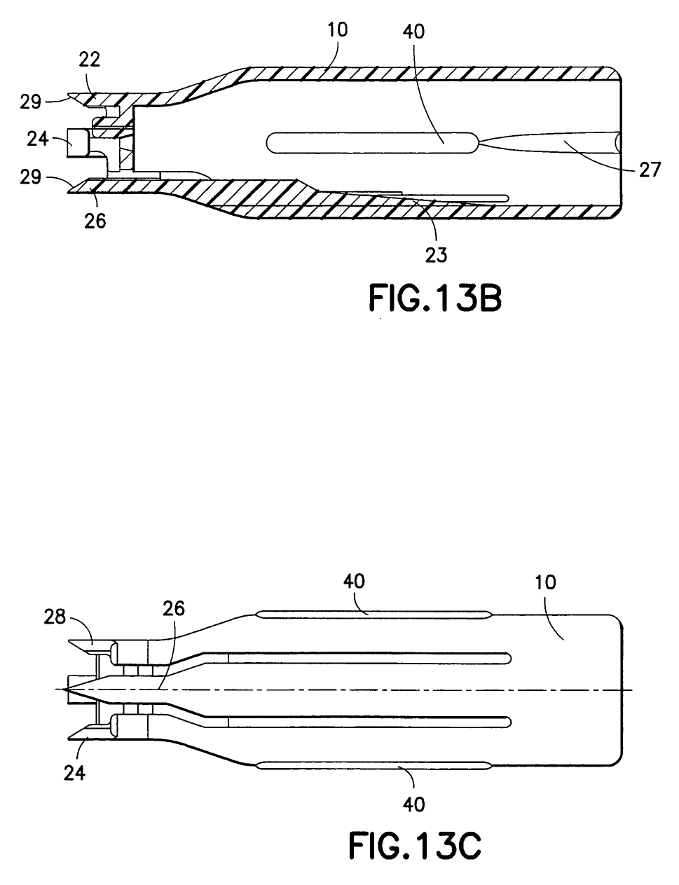

Within the circumference of the projections 22, 24, 26 and 28, the dilator housing 10 is substantially closed at the distal end by a member 34 to capture the spring 20. As shown, the projections 22, 24 and 28 can be joined at some point thereby creating a circular extension from the dilator housing 10. The member 34 can be provided at some point within this circular extension as desired by the application and provides at least one opening through which the injection needle 30 can extend, and is separated or cut from the cantilevered projection 26. Further the member 34 can provide a mounting slot 36 in which to secure the cutting member 32. The cutting member 32 can be staked or glued in the mount slot 36. Although a mounting slot 36 is shown in the exemplary embodiment, in yet other embodiments of the present invention, a single-sided mount surface or other securing mechanism can be used with which to secure the cutting member 32 to the dilator housing 10. the mounting slot can also comprise a blade stop for depth control as described in greater detail below. A more detailed illustration of the dilator housing 10 of the dilator sub-assembly 100 is shown in FIGS. 13A-13C.

FIG. 13A is a front view of the dilator housing 10, FIG. 13B is a cross-sectional view of the dilator housing 10, and FIG. 13C is an elevational view of the dilator housing 10. As shown in FIG. 13A, the projections 22, 24 and 28 can be joined at some point thereby creating a circular extension from the dilator housing 10, and projection 26, or the dilating foot, is shown at a position completing the circular extension and also being separated from the dilator housing 10 to permit the movement of the dilating foot when urged to do so. Also, as shown in FIG. 13A, the anchor point foot 22 is provided opposite the dilating foot 26. The cutting member 32 is also provided opposite the dilating foot 26, and the injection needle 30 is provided to extend form a point between the cutting member 32 and the dilating foot 26.

FIG. 13B illustrates an exemplary position of one stroke window 40, and one stroke window lead-in area 27. During assembly of the device as described in greater detail below, each cam stop 17 of the cam piece 16 is guided into a respective stroke window 40 using the stroke window lead-in area 27. Once in position within the stroke window 40, the cam stop 17 is captured, thereby slidably securing the cam piece within the dilator housing 10 of the dilator sub-assembly 100. FIG. 13B further illustrates the cam follower 23 of the dilator housing 10. During assembly of the device, the cam follower clearance slot 13 of the cam piece 16 receives the cam follower 23 to ensure alignment of the cam pip and the dilating foot 26. FIG. 13C illustrates an elevational view of one side of the dilator housing 10 and shows the separation of the dilating foot 26 from the dilator housing 10 in greater detail.

As noted above, at least one of the projections 22, 24, 26 and 28 that secure points of the conjunctival layer is cantilevered to be movable away from the injection site and the incision created by the cutting member 32. In the exemplary embodiment shown, projection 26 is cantilevered to be movable away from the injection site and the incision created by the cutting member 32. The projection, or dilating foot 26, includes an inclined inner surface, or ramp 38, which is engaged by the cam lip 19 of the cam piece 16 during movement of the syringe body 18, needle hub 12 and cam piece 16 toward the injection site within the dilator housing 10, such that the ramp 38 of the dilating foot 26 contacts the rounded cam lip 19 of the cam piece 16 and is gently deflected away from the injection site and the incision, thereby displacing the secured point of the conjunctival layer away from the incision of the cutting member 32 and creating a window opening in the conjunctival layer through which the intravitreal injection needle 30 can then enter and from which the injection needle can be retracted. That is, as shown in greater detail in FIG. 17, an opening 44 in the conjunctival layer of the eye 45 is provided substantially defined by the width of the incision 32a created by the cutting member 32 on one side, secured at points 24a and 28a by projections 24 and 28, and the displaced distance of point 26a of the deflected projection 26 at the opposite side. The injection entry of needle 30 can then occur at 30a. In an exemplary embodiment of the present invention, the deflected projection 26 can be moved a distance of between 0.1 mm and 4.0 mm from the relaxed state position, but is not limited thereto.

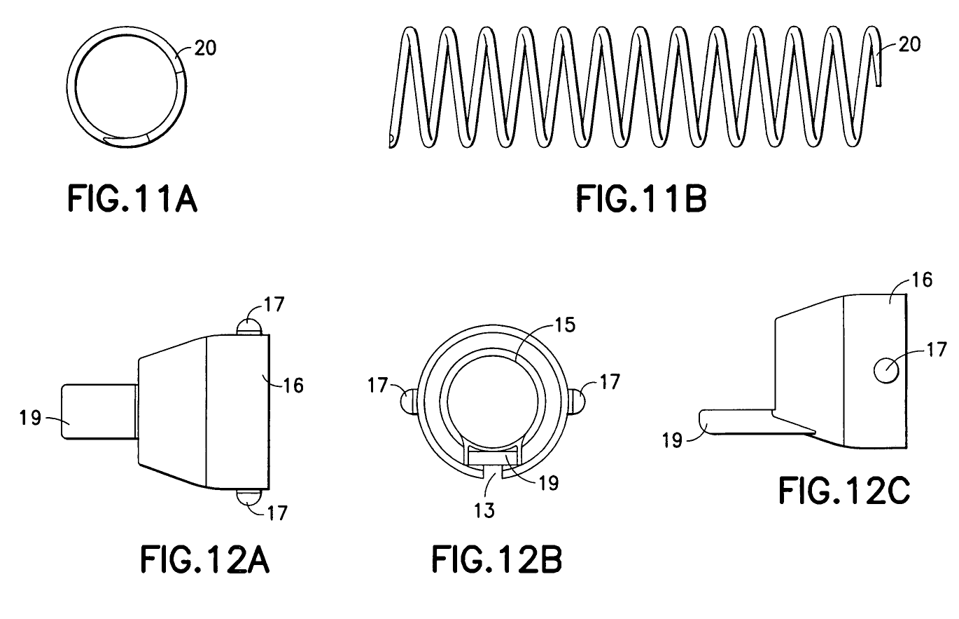

The dilator sub-assembly 100 further comprises the spring 20 that is captured between the member 34 and the hub 12, and which passes though the inner bore of the cam piece 16, such that the slidable movement of the syringe body 18, needle 30 and needle hub 12, and cam piece 16, toward the injection site within the dilator housing 10 is gently opposed. The spring 20 can comprise any suitable spring or urging mechanism, such as the coil spring shown in the exemplary embodiment. In the case of a coil spring, a portion of the needle hub 12 can be used to align and guide the spring within the dilator housing 10, and can be further used to secure one end of the spring in cooperation with the cam piece 16. An exemplary embodiment of the coil spring 20 is shown in greater detail in FIGS. 11A and 11B. FIG. 11A is a front view of the spring, and FIG. 11B is a side view of the spring. In an exemplary embodiment of the present invention, the spring 20 can comprise a stainless steel compression coil spring having an outside diameter of 0.210 inches, a free length of 1.00 inches, and can be constructed of wire having a diameter of 0.016 inches, but is not limited thereto.

Upon release of pressure upon the syringe body, the spring 20 urges the reverse movement of the syringe body 18, needle 30 and needle hub 12, and cam piece 16, away from the injection site within the dilator housing 10. When the syringe body 18, needle 30 and needle hub 12, and cam piece 16, are relaxed and moved away from the injection site by the spring 20, the ramp 38 of the dilating foot 26 is released from contact with the cam lip 19 of the cam piece 16, and gently returns to the non-deflected position, thereby returning the secured point of the conjunctival layer toward the incision and closing the window opening in the conjunctival layer.

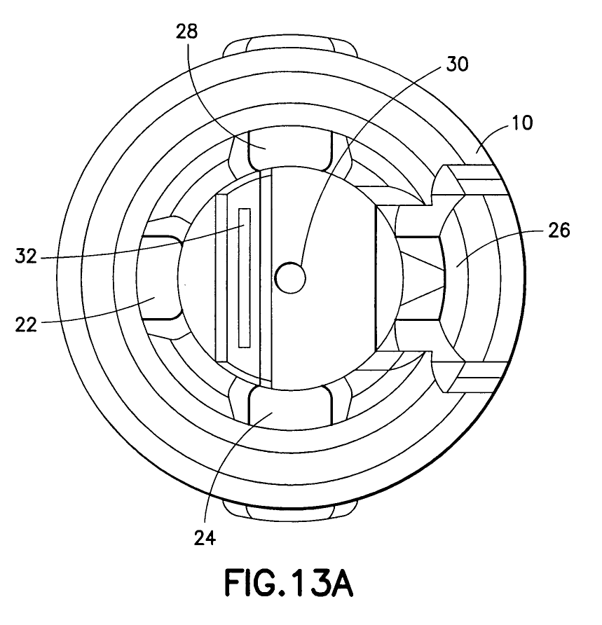

As noted in the above description, the projection, or dilating foot 26 includes an inclined inner surface, or ramp 38 which is engaged by movement of the cam lip 19 during advancement of the cam piece 16, syringe body 18, needle 30 and needle hub 12 toward the injection site within the dilator housing 10, such that the ramp 38 of the projection contacts the rounded cam lip 19 of the cam piece 16, and is gently deflected away from the injection site and the incision, thereby moving the secured point of the conjunctival layer away from the incision and creating a window opening in the conjunctival layer through which the intravitreal injection needle 30 then enters. The cam piece 16 can comprise a collar-like piece slidably disposed within the dilator housing 10 and into which the needle hub 12 can be secured. As shown in greater detail in FIGS. 12A-12C, the cam piece 16 can be configured as a substantially circular member to be fit within the dilator housing 10 and through which the spring 20 can pass, and to which the needle 30 and needle hub 12 can be secured, such as through a friction fit or other means. The cam piece 16 can further comprise a tapered surface at the distal end to avoid contact with the reduced diameter of the dilator housing 10 at points closer toward the distal end of the dilator housing.

FIG. 12A is a top view of an exemplary embodiment of the cam piece 16, and FIGS. 12B and 12C are front and elevational views of the exemplary embodiment of the cam piece 16, respectively. As shown in FIGS. 12A-12C, the cam piece 16 can comprise one or more detents or cam stops 17 on an outer surface that slidably fit within stroke windows 40 of the dilator housing 10 (see FIG. 7), and which restrict travel of the cam piece 16 within the dilator housing 10 once assembled. In the exemplary embodiment shown, two cam stops 17 for respective stroke windows 40 are shown, but is not limited thereto. Any number of stroke windows and cam stops can be used as desired by the application.

The cam piece 16 further comprises a compression spring clearance hole 15 which is provided to allow the spring 20 to pass through the inner opening of the cam piece 16. The cam piece 16 still further comprises the rounded cam lip 19 extending from the cam piece 16, and a cam follower clearance slot 13. As noted above, the dilator housing 10 comprises at least one stroke window 40, and at least one stroke window lead-in area 27. During assembly of the device, each cam stop 17 of the cam piece 16 is guided into a respective stroke window 40 using the stroke window lead-in area 27. Once in position within the stroke window 40, the cam stop 17 is captured, thereby slidably securing the cam piece within the dilator housing 10 of the dilator sub-assembly 100.

Once the dilator sub-assembly 100 is assembled with the syringe, the syringe body 18, needle 30 and needle hub 12, and cam piece 16, can be slidably moved within the dilator housing 10 as directed by the cam stops 17 of the cam piece 16 traveling in the stroke windows 40 of the dilator housing 10, and as directed by the cam follower 23 of the dilator housing 10 traveling in the cam follower clearance slot 13 of the cam piece 16. The cam stops and stroke windows both guide and limit the travel of the syringe body 18, needle 30 and needle hub 12, and cam piece 16, in both proximal and distal directions, and keep at least the cam piece 16 from falling free of the open end of the dilator housing 10 when no syringe is affixed. The spring 20 urges the syringe body 18, needle 30 and needle hub 12, and cam piece 16, in the reverse direction such that in a relaxed position, the cam stops 17 are at the farthest position in each respective stroke window 40. The cam stops and cam follower which guide the cam piece 16, further prevent rotation of the cam piece 16 within the dilator housing 10. Accordingly, once the needle hub 12 is secured in the cam piece 16, through a press fit or otherwise, the needle hub 12 also resists rotation, thereby facilitating assembly of the needle hub 12 with the syringe, such as in the case of a threaded fitting mechanism 14. The spring 20, cam stops 17, or combinations thereof, can also be used to limit the intravitreal injection depth.

The exemplary cam piece 16 is shown as a collar-like piece having an outside dimension of about 0.365 inches and a length of about 0.307 inches, for surrounding the needle hub 12, and having cam stops 17 on an outer surface for slidable fitting yet captured within stroke windows 40 in the dilator housing 10 to allow guidance within the dilator housing 10 and prevent over-travel in one or both directions. The exemplary cam piece 16 further comprises the cam lip 19 aligned with and extending toward the ramp 38. The cam lip 19 is about 0.128 inches wide and extends about 0.183 inches from the collar of the cam piece 16, and comprises a radius tip provided as the engagement mechanism for the ramp 38. The cam follower clearance slot 13 is provided adjacent to the cam lip 19 and is positioned to slidably receive the cam follower 23 of the dilating foot 26 of the dilator housing 10. In an exemplary embodiment of the present invention, the cam piece 16 can be comprised of a white acetal material, but is not limited thereto.

The ramp 38, cam piece 16 and needle 30 are configured such that the needle 30 begins its approach toward the injection site at substantially the same time that the ramp 38 engages the cam piece 16 and begins to deflect the dilating foot 26. However, the ramp 38, cam piece 16 and needle 30 are configured such that the dilating foot 26 creates the window opening in the conjunctival layer prior to the needle 30 reaching the injection site. In doing so, exemplary embodiments of the present invention substantially guarantee that the needle 30 will not contact the conjunctival layer prior to, during or after the intravitreal injection. Further, exemplary embodiments of the present invention substantially guarantee that the conjunctival layer will not be present at the intravitreal injection site during the intravitreal injection.

Once the needle 30 is in place, the plunger of the syringe is activated completing the intravitreal injection. When the syringe body 18, needle 30 and needle hub 12, and cam piece 16, are relaxed and moved away from the injection site, the ramp 38 of the dilating foot 26 is released by the cam lip 19 of the cam piece 16, and the dilating foot 26 gently returns to the non-deflected position, thereby returning the secured point of the conjunctival layer toward the incision and closing the window opening in the conjunctival layer. The incision can then close as a result of the normal healing process.

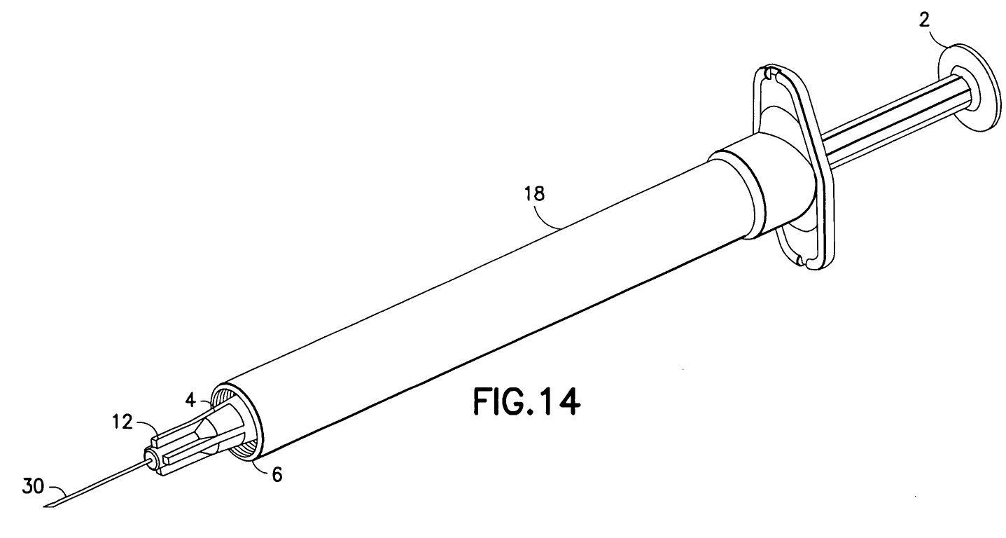

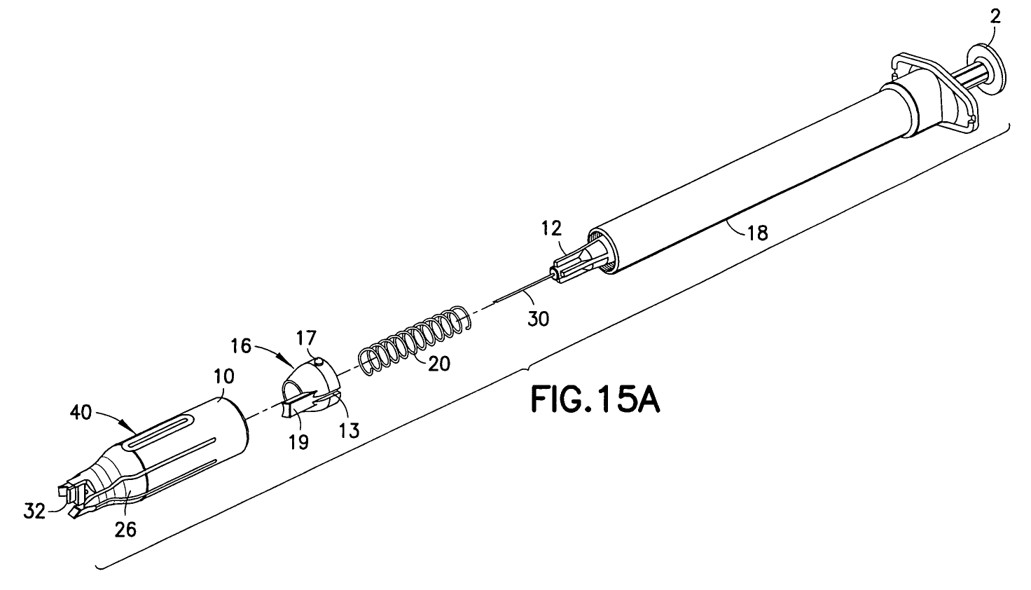

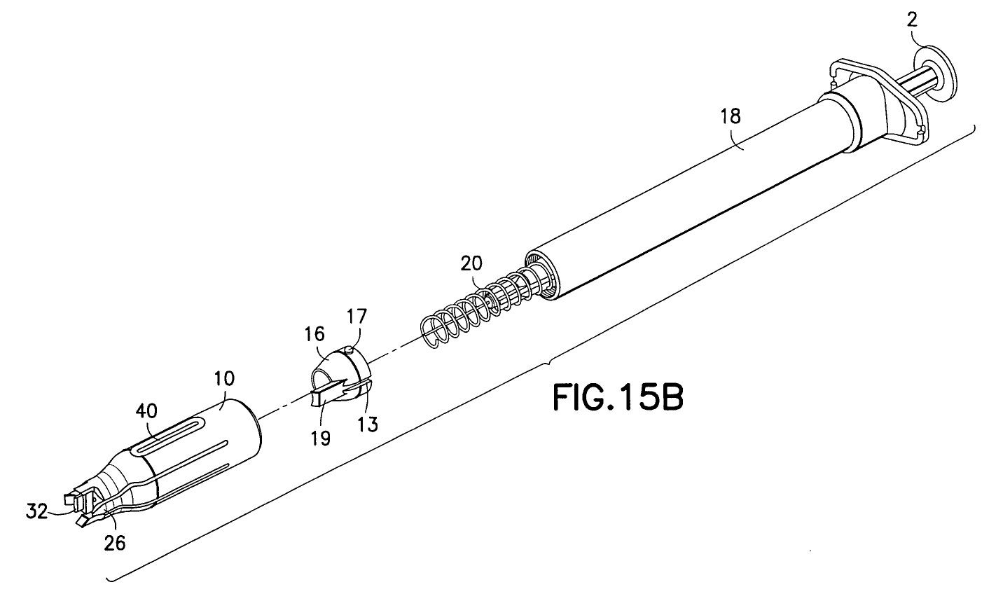

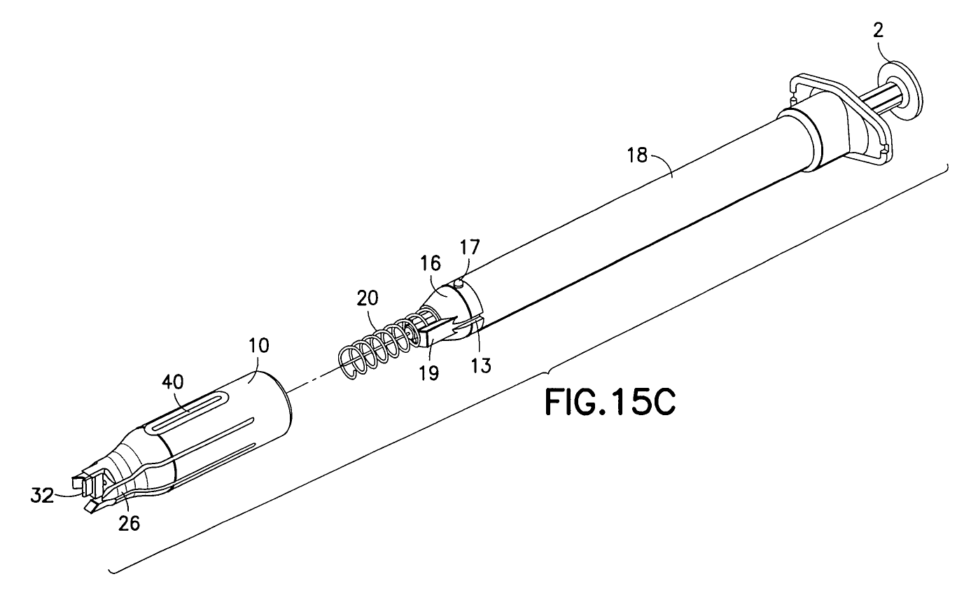

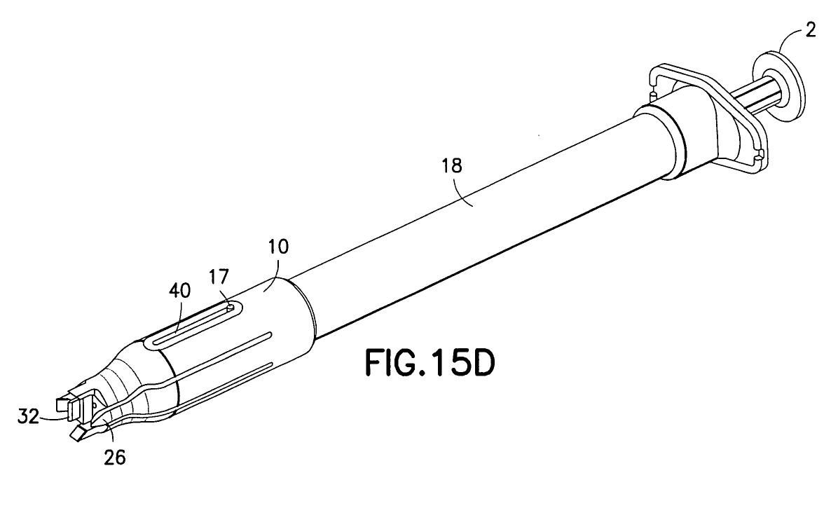

An exemplary assembly sequence will now be described in grater detail. FIG. 14 is a perspective view of a syringe to which the dilator sub-assembly 100 configured to perform as a conjunctival dilator can be attached, and FIGS. 15A-15D are perspective views illustrating an exemplary assembly of the syringe with the dilator sub-assembly 100 configured to perform as a conjunctival dilator.

In FIG. 14, the syringe 18 is shown with an assembled hub 12, including a hub taper 4, and injection needle 30. At an opposite end, a plunger 2 is provided. A leading edge 6 of the syringe body 18 is also provided which is assembled to slidably enter the open end of the dilator housing 10. In FIGS. 15A-15D, the blade 32 has already been assembled onto the dilator housing 10 as a sub-assembly into which the cam 16, and spring 20 can be inserted. It is also possible to assemble all components onto the needle/hub, and then attach the needle/hub onto the syringe barrel.

As shown in FIG. 15B, the compression spring 20 can be placed onto the needle/hub. The spring's inside diameter slips over the hub's ribs, and then presses onto the hub's taper 4. The spring's outside diameter is forced out to a larger diameter as a result of the hub's taper 4 which can serve to gently secure one end of the spring 20 to the hub 12.

The cam piece 16 can then be slipped over the spring 20 as shown in FIG. 15C, passing over the hub's tapered area, where it presses over the spring's increased diameter which can serve to gently secure the cam piece 16 to the hub 12. It is also possible to epoxy this sub-assembly.

The dilator housing 10 is then slipped over the spring 20 as shown in FIG. 15D. Preferably, fixturing is configured to ensure that the needle's tip does not hit any of the dilator housing's inside geometry during assembly. The dilator housing 10 is preferably indexed such that the dilating foot 26 is in-line with the cam's lip 19. The two cam stops 17 enter the dilator's cam stop or stroke window lead-in areas 27. As the dilator housing 10 is advanced, the dilator's cam stop lead-in is forced out until the cam stops 17 snap into the dilator's stroke window 40. Further, as described above but hidden from view in FIGS. 15A-15D, the cam follower clearance slot 13 provided adjacent to the cam lip 19 can slidably receive the cam follower 23 of the dilating foot 26 of the dilator housing 10.

An exemplary implementation of the present invention is the performance of intravitreal injections and/or targeted injections into the back of the eye. Such intravitreal injections can consist of but is not limited to, bevacizumab (Avastin), ranibizumab (Lucentis), pegaptanib sodium (Macugen), triamcinolone acetonide (Kenalog), ganciclovir, dexamethasone, cidofovir, Fomivirsen (Vitravene), voriconazole, amphotericin B, foscarnet, hyaluronidase (Vitrase), antibiotics, steroids, and others, including many in pharmaceutical/clinical development.

To do so, the intravitreal injection needle 30 can comprise a 27 ga to 34 ga needle, wherein larger needles can be provided to inject drugs with crystalline particles, and smaller needles can be used to inject drugs in solutions. In an exemplary embodiment of the present invention, the needle 30 can be extended from the device a distance of between about 0.025 mm and 0.200 mm. Further, specific needle bevels, surface finishing, and/or other needle configurations can be used to provide desired results.

Examples of the deflection and needle insertion described above are shown in regard to the following. Specifically, an exemplary use of the embodiment of the present invention comprises operations which, preferably, first prepare the syringe 18 and dilator sub-assembly 100 and then place the dilator sub-assembly 100 against a targeted intravitreal injection site. The user can then press the syringe 18 downward against the targeted intravitreal injection site to cut the conjunctival layer upon contact using the cutting member 32, continue pressing the syringe against the targeted intravitreal injection site to cam the cantilevered dilating foot 26, and advance the injection needle 30 into the intravitreal injection position, activate the plunger of the syringe to complete the injection, and then withdraw the device. A subsequent step for shielding and/or preventing re-use of the device can also be provided.

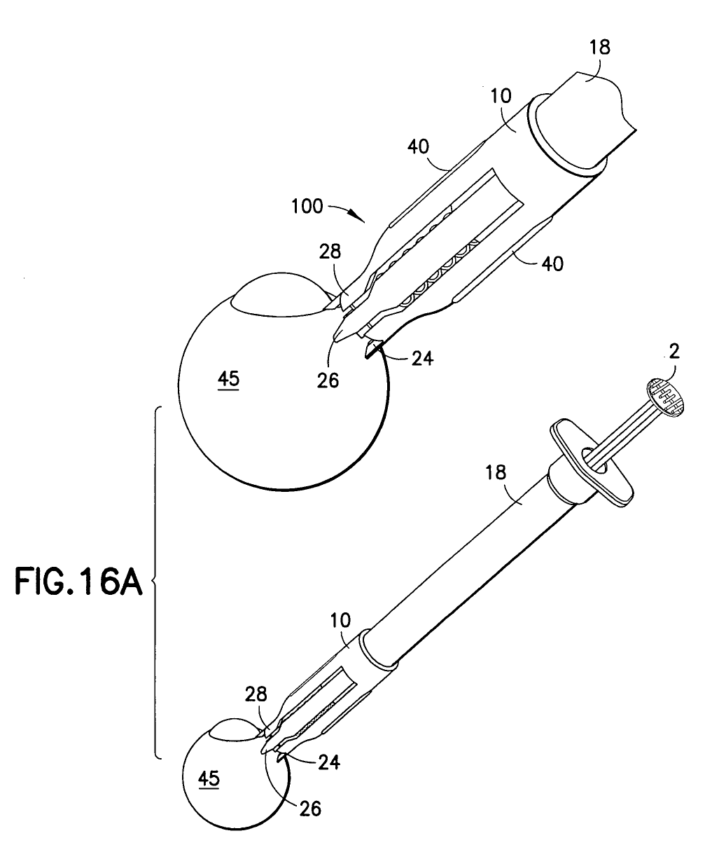

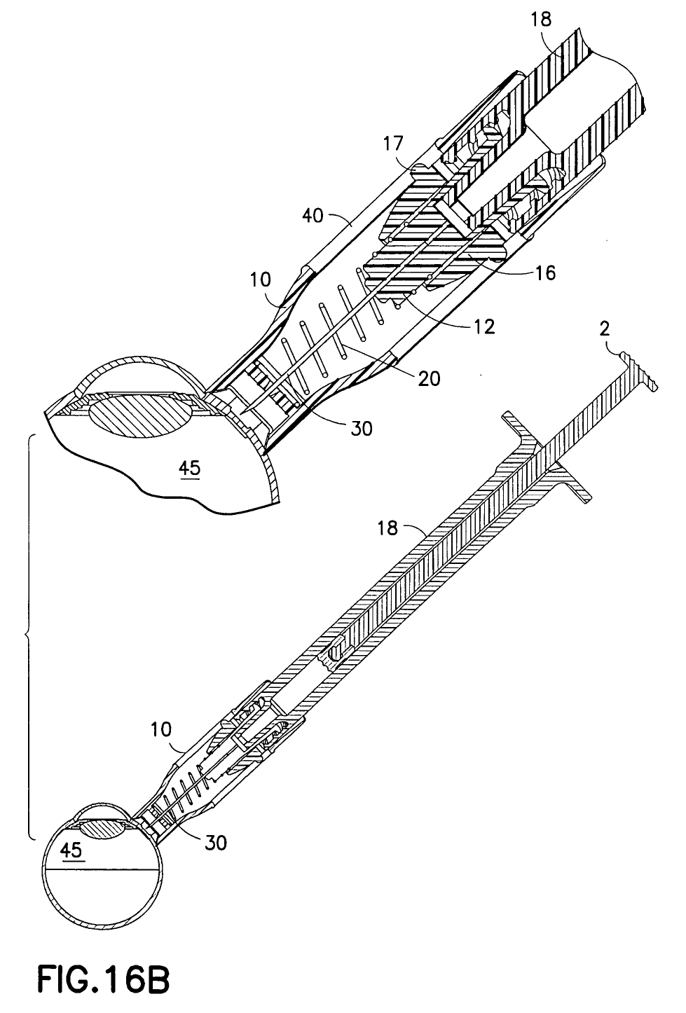

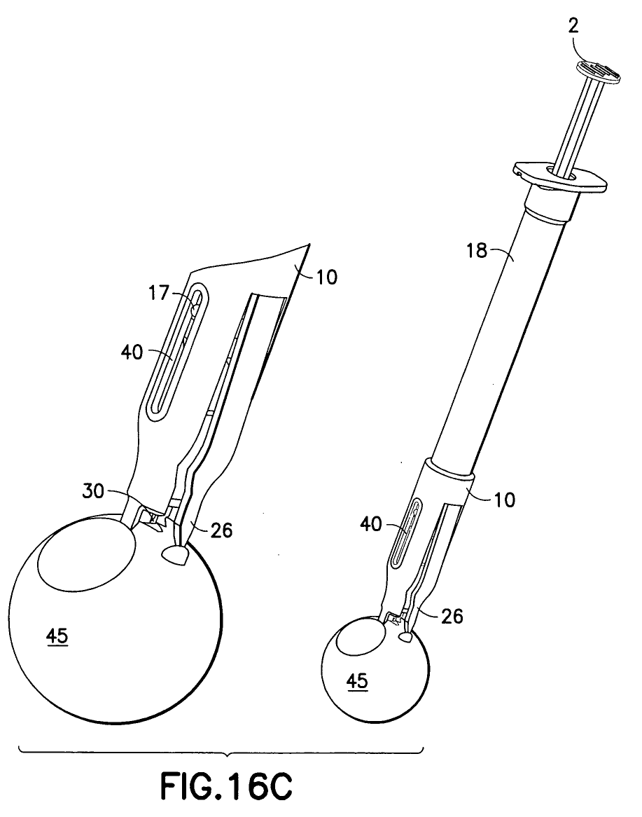

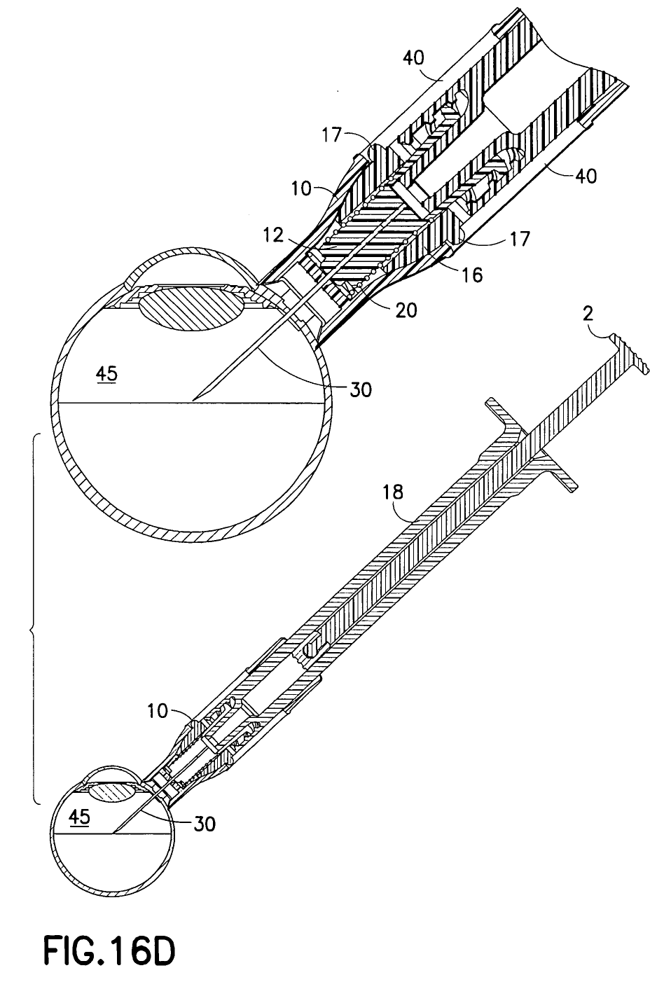

In FIG. 2, the intravitreal injection device is shown in the relaxed and deactivated state with the needle 30 retracted and the cam piece 16 at the bottom of the ramp 38 of the dilating foot 26. That is, the dilating foot 26 is shown in the relaxed state. While in this state, a user can grasp the intravitreal injection device firmly by the syringe body 18, and place the proximal end at the desired intravitreal injection site (see also FIG. 16A). Once placed at the desired site, the user can press the syringe body 18 toward the intravitreal injection site. This secures each point of the conjunctival layer using the projections 22, 24, 26 and 28. This also brings the cutting member 32 into contact with the conjunctival layer to provide the desired incision through the conjunctival layer (see also FIGS. 16B and 16C).

The projections 22, 24, 26 and 28 can be provided to have a diameter (defined by the arcuate projection ends) coaxial with the injection needle 30. Each projection 22, 24, 26 and 28 can have a tapered, pointed, or rounded end, to facilitate securing the points of contact of the conjunctival layer without cutting or damage to the conjunctival layer. By securing the points of contact of the conjunctival layer, exemplary embodiments of the present invention ensure that the opening is provided in the conjunctival layer by the movement of one or more projections. That is, as each projection is securing a point of contact of the conjunctival layer, a movement of the dilating foot 26 can be used to move that point of contact. By combining a displaced point of contact with the small incision created by the cutting member 32, at opposite sides of a desired injection site, embodiments of the present invention provide an efficient and effective method for creating a controlled opening in the conjunctival layer for injection.

Further, as shown in FIGS. 2 and 3, the cutting member 32 is provided within the circumference of the projections 22, 24, 26 and 28 to gently contact and incise the conjunctival layer upon the initial contact. In an exemplary embodiment of the present invention, the cutting edge of the cutting member 32 extends to a distance substantially equal to the distance of the projections 22, 24, 26 and 28, although not limited thereto. The cutting edge of the cutting member 32 can extend to a greater distance or a lesser distance from the blade stop of the blade slot as desired by the application.

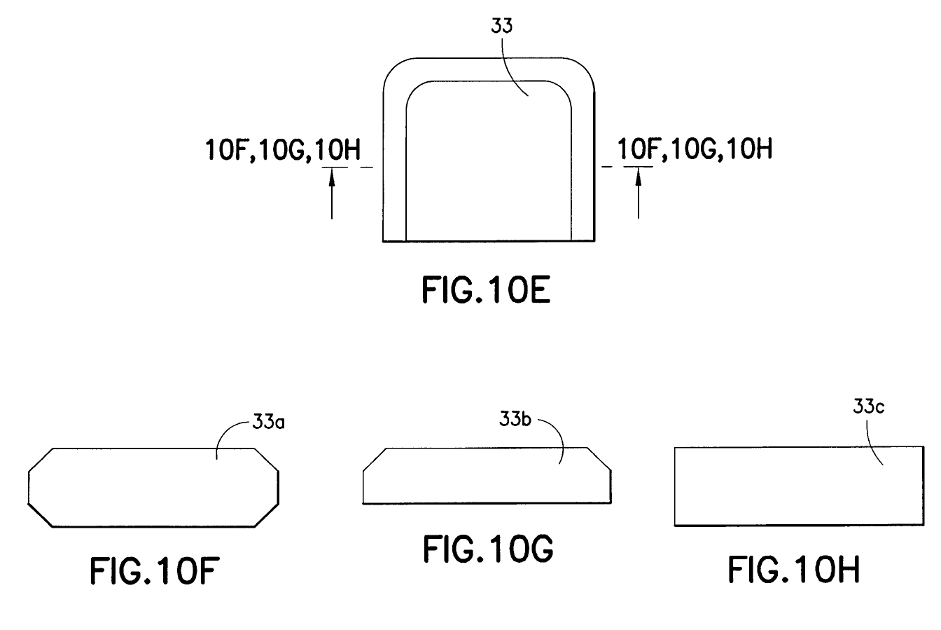

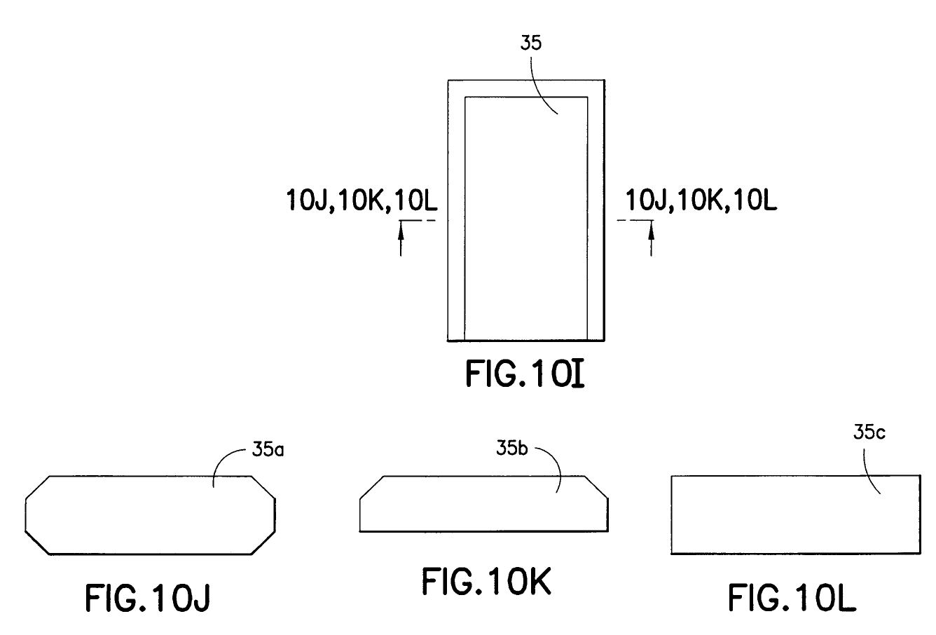

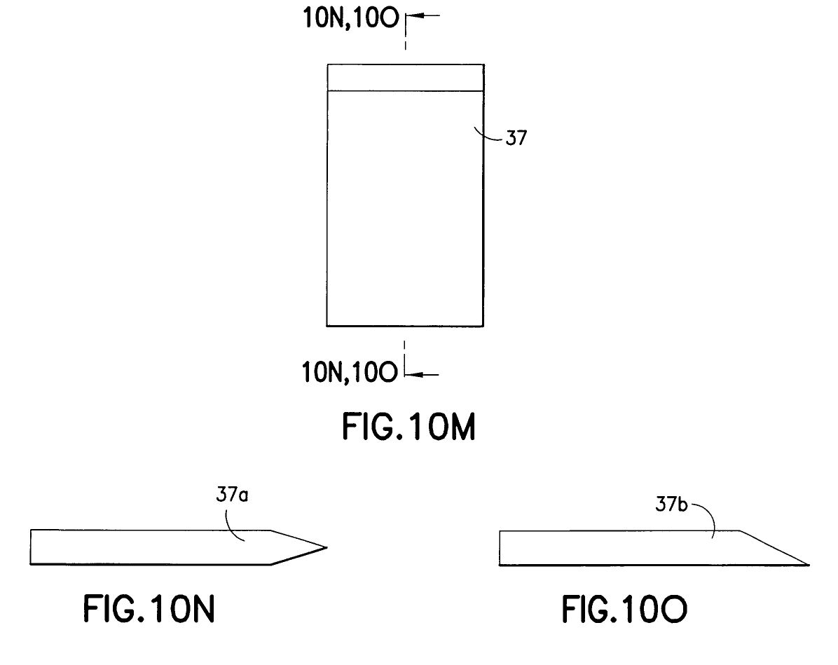

The cutting member can have a width between 0.5 mm and 4.0 mm, and can be comprised of any number of suitable metal or plastic blades, including but not limited to metal, diamond, silicon and sapphire. The cutting member 32 can have any number of suitable shapes, including but not limited to chisel, slit, stab and crescent shapes, and can include any number of suitable blade bevel geometries, including but not limited to single or double bevels, variable bevel angles and bevel lengths. In an exemplary embodiment of the present invention, the cutting member is a stainless steel blade configured as shown in FIGS. 10A-10D, including a double bevel, a thickness of 0.009 inches, a width of 0.125 inches, and a length of 0.195 inches. FIG. 10A shows a perspective view of an exemplary embodiment of the blade 32, and FIGS. 10B, 10C and 10D show top, back and side views of an exemplary embodiment of the blade 32, respectively.

Other blade geometries that may be used include the exemplary embodiments shown in FIGS. 10E-10O. A top view of an exemplary crescent shaped blade 33 is shown in FIG. 10E, an appears substantially as a chisel blade with rounded corners instead of sharp corners, and having either single or double bevels on the sides of the blade. FIGS. 10E-10H are cross-sectional views of the exemplary crescent shaped blade and illustrate sides having double bevels in FIG. 10F, sides having a single bevel in FIG. 10G and sides with no side bevels in FIG. 10H. A top view of an exemplary chisel blade 35 is shown in FIG. 10I, with either a single bevel (i.e., a bevel on the cutting edge either on the top or on the bottom face of the blade), or a double bevel (i.e., a bevel on the cutting edge on both the top and bottom face of the blade), and with either a single or double bevel on the side of the blade. FIGS. 10J-10L are cross-sectional views of the exemplary chisel shaped blade and illustrate sides having double bevels in FIG. 10J, sides having a single bevel in FIG. 10K and sides with no side bevels in FIG. 10L. A top view of an exemplary chisel blade 37 is shown in FIG. 10M, with either a single bevel as shown in FIG. 10O (i.e., a bevel on the cutting edge either on the top or on the bottom face of the blade), or a double bevel as shown in FIG. 10N (i.e., a bevel on the cutting edge on both the top and bottom face of the blade).

The incision of the conjunctival layer is accomplished by the cutting member 32, and the flexible and pliable conjunctival layer once closed after injection, safely regrows and heals. Selection of the cutting member 32 and the associated depth of the incision can be configured based upon a conjunctival layer thickness of around 70-120 microns, and can be further configured to minimize edema of the conjunctival layer. In the exemplary embodiment of the present invention, the depth of the incision is substantially set by the cutting member 32 length beyond the blade stop, the projections 22, 24, 26 and 28 surrounding the cutting member, and the curvature of the eye. In this or other embodiments of the present invention, the blade stop provided by the mount surface 36 can act as a depth limiter for the incision made by the cutting member 32.

As shown and described above, exemplary embodiments of the present invention ensure that the projections 22, 24, 26 and 28, and the cutting member 32, are the only portions of the intravitreal injection device which contact the conjunctival layer. Further, the projections 22, 24, 26 and 28, and the cutting member 32 are not used to penetrate any remaining surfaces or provide intravitreal injection functions. In doing so, risks of infection due to contact with the intravitreal injection are substantially reduced.

As noted above, the gripped syringe body 18 can be gently slid toward the injection site and into the stationary dilator housing 10. The spring 20 captured between the member 34 and the hub 12, gently opposes the slidable movement of the syringe body 18, needle 30 and needle hub 12, and cam piece 16, toward the injection site within the dilator housing 10. Continued pressure will result in the movement of the syringe body 18, needle 30 and needle hub 12, and cam piece 16, toward the injection site, such that the ramp 38 of the projection contacts the cam lip 19 of the cam piece 16, and the dilating foot 26 is gently deflected away from the injection site and the incision, thereby moving the secured point of the conjunctival layer away from the incision and creating a window opening in the conjunctival layer through which the intravitreal injection needle 30 then enters.

The hub assembly 10 can be coupled with any suitable syringe. That is, the cam piece 16 can be secured to any number of hubs, which in turn, can be secured to any number of suitable syringes. FIG. 5 and the assembly steps of FIGS. 15A-15D illustrate an exemplary coupling of the dilator sub-assembly 100 with a Luer-Lok™ syringe, but embodiments of the present invention are not limited thereto. The fitting mechanism 14 of the dilator sub-assembly 100 can comprise a Luer-Lok™, threaded or slip fitting, or any other suitable fitting mechanism as desired by the application.

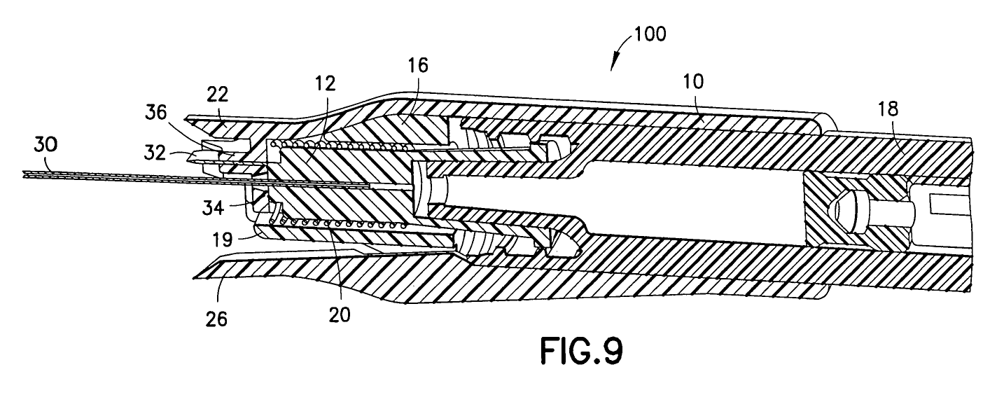

FIG. 6 illustrates an exemplary intravitreal injection device during activation. As shown in FIG. 6, the spring 20 is being compressed and the cam lip 19 of the cam piece 16 has moved up the ramp 38 and has forced the dilating foot 26 to move laterally away from the blade 32 (see also FIG. 16B). The dilator housing 10 remains stationary against the intravitreal injection site, and the needle 30 is beginning to emerge. As illustrated in FIGS. 7 and 9, the intravitreal injection device has been fully activated, with the spring 20 fully compressed, the needle 30 fully exposed out of the dilator housing 10, and the cam lip 19 of the cam piece 16 moved up past the ramp 38, which is now shown in contact with the contoured leading edge of the cam piece 16 (see also FIG. 16D). The plunger barrel of the syringe can now be pushed forward to dispense the medication.

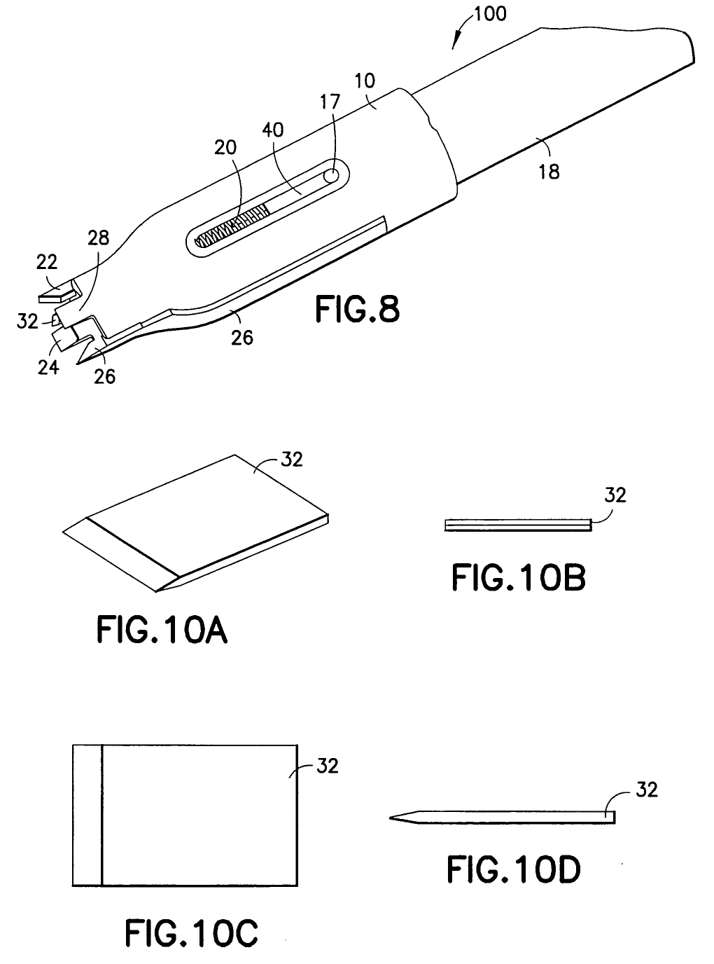

In yet other embodiments of the present invention, access through one or more parts of the dilator housing 10 can be provided to determine a status of the mechanisms therein, such as the needle position, cam position and so forth. FIG. 8 illustrates the stroke windows 40 through which a user can detect needle position or other features within the dilator housing 10. In yet other embodiments of the present invention additional visibility windows or larger visibility windows can be provided at the position shown, or at yet other positions as desired by the application.

In an exemplary use shown in FIGS. 16A-16D, the dilator sub-assembly 100 of the intravitreal injection device, into which a needle is assembled, is attached to a standard Luer-Lok™ syringe 18 filled with medication. The syringe 18 is filled with the intravitreal medication though such steps as aspirating medication from a vial, or is pre-filled with the medication from a supplier. The filled syringe 18 is then connected to the dilator sub-assembly 100 via the connection mechanism 14, in this case, a Luer-Lok™ the distal end of the syringe 18. Dead space in the fluid path is cleared by pushing the syringe distally such that a small amount of medication exits the distal end of the needle 30 tip.

The patient's scleral conjunctiva is cleaned, anesthetized, and prepared via any number of standard procedures. The user then engages the device by placing the dilator sub-assembly 100 against the injection site and applying initial pressure using the syringe body to incise the conjunctiva. The exemplary embodiments of the present invention allow either a one handed or two handed activation technique to be used with equal applicability. The user places the device such that the one of the two dilator's stationary feet, that is adjacent to the dilating foot, is tangent to the eye's limbus. While gripping the syringe barrel, the user then starts advancing the syringe body toward the eye and into the dilator sub-assembly 100. The blade contacts and incises the conjunctiva until the dilator's blade stop contacts the eye. That is, the user places the intravitreal injection device onto the cleaned area of the conjunctiva as shown in FIGS. 16A and 16B, and gripping the syringe body, places enough pressure against the injection site such that the cutting member 32 incises the conjunctival layer on the sclera and the projections 22, 24, 26 and 28 act as anchor points.

Next, conjunctival dilating occurs. Advancing the syringe body further will start the dilating foot's motion. With the dilator sub-assembly blade stop and all feet engaged, the dilator sub-assembly will no longer move forward. The cam lip and the syringe barrel's leading edge will start engaging the cam follower surfaces of the dilating foot. The cam mechanism will fully advance the dilating foot 26 away from the blade 32 prior to the needle 30 touching the conjunctiva. This will fully dilate the incision created during the initial engagement. That is, as the intravitreal injection device is pushed onto the eye as shown in FIG. 16C, the spring 20 compresses, the dilator housing 10 remains stationary and the cam lip 19 of the cam piece 16 rides up the ramp 38 of the cantilevered and deflectable dilating foot 26, causing the dilating foot to move laterally in a direction opposite to that of the anchor point as shown. As the dilating foot moves, it spreads the conjunctival tissue away from the anchor point. The result is a bare “scleral window” being exposed with no conjunctival tissue covering it.

As the syringe body 18, needle 30 and needle hub 12, and cam piece 16, move further proximally, the needle 30 is exposed and penetrates the scleral window to enter the vitreal chamber of the eye. The user can visualize the cam position and, correspondingly, the needle position, by observing the positions of the cam stops 17 through the visibility window, or stroke windows 40.

Next, full stroke will be described. Advancing the device past the gaping position will lead to the needle 30 penetrating the sclera through the dilated conjunctiva. Full stroke occurs when the hub bottoms out on the dilator and the cam stop 17 reaches the distal end of the stroke window 40. Once full stroke occurs, the user can advance the syringe plunger to inject the medication into the eye.

The user can then remove the device by pulling it straight out of the eye. That is, the user can first remove pressure applied to the device such that the needle 30 will retract out of the clean open widow. The compression spring 20 will force the dilator to return to its initial position by releasing the cam mechanism. As the dilating foot 26 returns to the initial position, the conjunctiva window is closed to cover the needle puncture site, and returned to its initial position. The needle 30 then retracts back into the dilator sub-assembly. That is, upon removal of the intravitreal injection device from the injection site, the dilating foot 26 is released by movement of the syringe away from the injection site such that the dilating foot gently returns toward the injection site and the incision, thereby moving the secured point of the conjunctival layer toward the incision and closing the window opening in the conjunctival layer.

In yet other embodiments of the present invention, a re-use lock can be provided to prevent re-use of the syringe. Further, caps, safety shields or other techniques can be provided to shield the needle and/or blade before and after use.

In still other embodiments of the present invention, the projections 22, 24, 26 and 28 or any other contact surface can incorporate pads 29 soaked with betadine, antibiotics, anesthetics and/or other desired substances to further minimize risks associated with intravitreal injections.

As noted above, a benefit of the exemplary embodiments described above is that the arrangement of the projections 22, 24, 26 and 28, once properly placed for intravitreal injection, assists the user in targeting the injection. In an intravitreal injection, the injection should preferably be targeted, for example, to avoid damage to the lens of the eye, to avoid direct injection upon the retina, and so forth. To aid in doing so, at least one of the projections can be used to identify the desired intravitreal injection site. For example, as shown in FIG. 17, the projections and cutting member 32 can have a foot-to-blade distance such that the projection 28 can be placed at a specific point 28a, such as at the limbus 42, so the scleral window 44 and intravitreal injection 30a are created at a desired location, slightly posterior to the limbus 42 (i.e., 3 mm posterior to the limbus). FIG. 17 is an illustrative view of an injection site showing exemplary positions, movements and openings created with the hub configured to perform as a conjunctival dilator. Such a feature eliminates the need for calipers or rulers currently used for such intravitreal injection measurements. Accordingly, the geometry of the distal end of the dilator sub-assembly 100 lends itself to acting as an intravitreous marker. The distance between the point 22a secured by the stationary foot 22 and the center axis of the needle entry 30a can be set to between 3.0 mm and 4.0 mm, and the stationary foot 24 or 28 parallel to the blade 32 can be then placed on the limbus 42 at point 28a and the injection at 30a will automatically be between 3.0 mm and 4.0 mm posterior to the limbus.

The circular geometry of the stationary feet at the distal end also serves to provide a stable “seating” of the device on the eye. Once the device is placed with the feet perpendicular to the ocular surface, the needle can advance coaxially and as a result, the needle will always enter and exit the eye perpendicular to the ocular surface. This is a departure from the conventional techniques in which the physician has no restrictions on the degrees of freedom available. As a result the needle may enter the eye at some undesired non-perpendicular angle to the ocular surface.

As noted above, in an exemplary embodiment of the present invention, a standard syringe with any suitable needle between 27 ga and 34 ga can be used but is not limited thereto, for drug delivery into the vitreal chamber. The syringe can either be pre-filled or can house drug aspirated from a vial. However, in a convention use, the needle first penetrates the conjunctival layer and then advances through all scleral layers before entering the vitreal chamber. As also noted above, one major concern with such conventional devices and techniques is the risk of endophthalmitis, a serious infection of the eye, due to the needle tracking through the heavily bacteria-laden conjunctival layer before entering the immunologically non-privileged vitreal chamber. The current incidence of endophthalmitis is 1 in 1000 injections, and as the frequency of intravitreous drug delivery increases, the cumulative incidence of endophthalmitis can reach 1 in 100. Bacteria entering the vitreal chamber through the pathway can increase risk of developing endophthalmitis, a serious infection of the eye.

Exemplary embodiments of the present invention provide an advantage over conventional devices because the dilator contacts the conjunctival layer and moves it aside such that the needle does not penetrate conjunctiva and potentially track bacteria all the way through to the vitreal chamber. In doing so, a sterile needle that has not contacted the conjunctiva will enter the vitreal chamber, thus potentially reducing the risk of endophthalmitis-causing bacteria entering the vitreal chamber. In scleral-approach cataract incisions, a “scleral peritomy,” in which the conjunctiva is cut and moved aside, is performed before creating a slit incision into the sclera. However, the exemplary embodiments of the present invention accomplish a similar act of moving the conjunctiva aside, but with fewer instruments and is an application for intravitreous injections that can be performed in the physician's office rather than an operating room.

As noted above, a solution to the needle tracking bacteria from the conjunctival layer through to the vitreal chamber is accomplished by first having the outer layers of the conjunctival layer cut and separated by the blade and dilated foot, rather than by the needle itself. A sterile needle can then pass through the sclera and never come into contact with any bacteria from the outer conjunctival layers. This preserves the sterility of the immunologically non-privileged vitreal chamber and can reduce the incidence of endophthalmitis caused by the injection procedure.

In yet other exemplary embodiments of the present invention, one or more parameters can be varied. For example, the distance from the blade to the movable foot can be varied to optimize the size of the conjunctival window through which the needle can pass without contacting the conjunctiva. The distance from the needle to the stationary foot parallel with the blade can also used to mark the injection site as measured by the distance from limbus. This distance can range from 3.0 mm to 4.0 mm depending on whether the tool is used on a patient that is phakic, pseudophakic, or aphakic. Still further, the diameter of the distal end, or the distance between one stationary foot and another can be varied to optimize the gripping efficacy of the stationary feet. The distance between the blade and needle can also be varied to accommodate for manufacturing constraints in assembling the blade into the dilator housing or to account for the size and shape of the conjunctival window resulting from the movable foot/blade interaction.

The blade can be made of any material currently used to create knives for ophthalmic surgery (e.g., metal, diamond, silicon, sapphire or similar materials). The shape of the blade can also be varied to optimize the sharpness, wound shape, and blade manufacturing efficiency. Potential blade shapes include, but are not limited to, chisel, slit, stab, and crescent shapes. Further, the blade bevel geometry can be varied to optimize the sharpness, wound shape, and blade manufacturing efficiency and can include a single bevel, double bevel, various bevel angles, and/or various bevel lengths. In doing so, the blade width can also be varied between 0.5 mm-4 mm.

The blade assembly area can be comprised of a slot (as shown) or an open faced slot (pocket). In each case, the blade can be retained therein with a press-fit, epoxy, or ultrasonic welding. For the pocket assembly, the blade can be secured using epoxy directly to the pocket face or held therein using a cap that can be ultrasonically welded or epoxied. A blade exposure length (i.e., an amount of blade exposed outside of the attachment ledge/slot) can be varied to optimize the efficacy of cutting the conjunctiva.

In these or any other exemplary embodiments of the present invention, the needle gage may span between 27 ga and 34 ga but is not limited thereto, and the length of needle exposure outside of the dilator housing can be varied between 0.025 mm and 0.2 mm. A variety of needle bevel geometries can also be used to optimize needle sharpness and drug delivery efficacy, and a variety of needle surface finishes can be applied. In still other exemplary embodiments of the present invention, the needle may comprise a sideport geometry in which the distal tip is closed and the drug can flow through a sideport opening. This would allow for the drug to be delivered at a slower flowrate and would prevent the drug delivery from being targeted at the retina.

The dilating foot can comprise a variety of shapes, and a variety of radii on the distal end of the foot to optimize the conjunctival window creation. The stationary feet can also comprise a variety of shapes, and a variety of radii on the distal ends of the feet to optimize the gripping efficacy. The distance to which dilating foot can extend from its relaxed state can also be varied to range between approximately 0.1 mm and 4 mm.

The exemplary embodiments of the present invention can further comprise an automatic disable feature upon needle retraction to act as a needle safety feature and force single use of the product. These and other exemplary embodiments can also comprise a safety shield on the blade to protect the blade and the user from accidental stick injuries. The safety shield can be further configured to lock upon activation to prevent reuse.

The size and shape of the visibility or stroke window can also be varied as desired to provide device stability and appropriate visibility to the user. Still other exemplary embodiment of the present invention can be uniquely configured to require either one-hand or two-handed activation, and the needle hub can be configured to connect either to a Slip Tip, Luer-Lok™, or any number of other syringe configurations. Such a syringe can be configured to contain volumes of between 0.05 mL and 1.0 mL, but is not limited thereto. The distal end of the syringe barrel can be configured to be compatible with a standard needle hub or otherwise. That is, a syringe with a distal end to accommodate a non-standard hub may be used.

Still further exemplary embodiments of the present invention can comprise one or more pads soaked with betadine and/or anti-infective and/or anesthetic (e.g., lidocaine drops or lidocaine jelly) substances or other materials on the distal end of the stationary feet. As the device is placed on the eye, the pressure placed on the device can be used to cause the liquid to seep out of the pads and onto the eye. The device would then operate as described above to create the injection into the eye. This would combine multiple steps of the procedure into one step through the use of one device, as there would be no need for multiple materials.

The above exemplary embodiments of the present invention are based in part upon the following technical principles. A “scleral peritomy” or “conjunctival peritomy” in which the conjunctiva is cut and moved aside to expose the bare sclera, is performed as part of a scleral approach to cataract surgery and in anesthesia delivery to the eye. The purpose of performing a scleral or conjunctival peritomy is to minimize bacterial contamination of the incision site.

In a scleral approach cataract surgery, a conjunctival peritomy is performed by cutting the conjunctiva with Westcott scissors. The conjunctiva and Tenon's capsule are dissected from the limbus using Westcott scissors and toothed forceps. A keratome is used to create a “scleral tunnel” incision in the bare sclera through which instruments are inserted for capsulorhexis, phacoemulsification, irrigation/aspiration, and intraocular lens implantation. At the end of the cataract procedure, the conjunctiva is pulled over the scleral tunnel incision and returned to its initial position. This may contribute to lower incidences of reported endophthalmitis.

A sub-Tenon's peribulbar block is a method of delivering anesthesia to the eye and is an alternative to the retrobulbar block. A button hole is made in the conjunctiva and Tenon's capsule using Westcott scissors and forceps. Blunt dissection of the conjunctival tissue is completed with Westcott scissors. An anesthetic-filled syringe with attached needle is inserted underneath Tenon's capsule and the anesthetic is injected.

Accordingly, through advancements, improvements, and unique and novel mechanisms, exemplary embodiments of the present invention provide a solution to the problem of needle tracking bacteria from the conjunctival layer through to the vitreal chamber by first having the outer layers of the conjunctival layer cut and separated by the blade and dilated foot, rather than by the needle itself A sterile needle can then pass through the sclera and never come into contact with any bacteria from the outer conjunctival layers. This preserves the sterility of the immunologically non-privileged vitreal chamber and can reduce the incidence of endophthalmitis caused by the injection procedure.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention and the following claims.

What is claimed is:

1. An injection device attachment for providing a coordinated cutting and spreading mechanism to provide an access window free of a conjunctival layer and through which an injection needle can be inserted, the device comprising:
a plurality of projections extending from a first end of said attachment configured to contact a conjunctival layer, comprising at least one projection configured to be displaced concurrently with a point on said conjunctival layer;
at least one cutting member extending from said first end of said attachment for incising said conjunctival layer; and
an injection needle hub and injection needle, wherein said injection needle hub and injection needle are configured to slide within an inner bore of said attachment and extend said injection needle from said first end of said attachment at a point between said at least one projection configured to be displaced and said cutting member, for insertion into an access window free of said conjunctival layer.

2. An injection device attachment as described in claim 1, wherein said plurality of projections extend from said first end of said attachment in a substantially circular pattern to secure points of a surface of a conjunctival layer, and concentric with said point from which said injection needle extends.

3. An injection device attachment as described in claim 2, wherein the cutting member can be provided to contact and incise said conjunctival layer within said circular pattern of said plurality of projections and adjacent to said point from which said injection needle extends.

4. An injection device attachment as described in claim 3, wherein said at least one projection is configured to be displaced concurrently with a point on said conjunctival layer in a direction away from the incision created by the cutting member to form said access window free of said conjunctival layer.

5. An injection device attachment as described in claim 1, further comprising:
a cam engaged with said injection needle hub and injection needle, wherein said cam is configured to contact said at least one projection configured to be displaced concurrently with a point on said conjunctival layer to displace said projection.

6. An injection device attachment as described in claim 5, further comprising:
at least one stroke window; and
said cam comprises at least one cam stop, wherein said cam stop is slidably disposed within said at least one stroke window and which limits a slidable travel of said cam, injection needle hub and injection needle.

7. An injection device attachment as described in claim 1, further comprising:
a spring disposed between said first end of said attachment and said injection needle hub and injection needle.

8. An injection device attachment as described in claim 1, further comprising:
a second end configured to slidably receive a syringe, wherein said injection needle hub and injection needle are configured to be removably secured to said syringe.

9. An injection device attachment as described in claim 1, wherein said at least one projection configured to be displaced concurrently with a point on said conjunctival layer is further configured to close said access window upon removal of said injection device from said conjunctival layer.

10. An injection device for providing a coordinated cutting and spreading mechanism to provide an access window free of a conjunctival layer and provide an intravitreal injection through said access window, the device comprising:
a dilator housing comprising a plurality of projections extending from a first end of said housing configured to contact a conjunctival layer, comprising at least one projection configured to be displaced concurrently with a point on said conjunctival layer and at least one cutting member extending from said housing for incising said conjunctival layer; and
a syringe comprising an injection needle hub and injection needle, wherein said syringe, injection needle hub and injection needle are configured to slide within an inner bore of said dilator housing and extend said injection needle from said dilator housing at a point between said at least one projection configured to be displaced and said cutting member for insertion into an access window free of said conjunctival layer.

11. An injection device as described in claim 10, wherein said plurality of projections extend from said housing in a substantially circular pattern to secure points of a surface of a conjunctival layer, and concentric with said point from which said injection needle extends.

12. An injection device as described in claim 11, wherein the cutting member can be provided to contact and incise said conjunctival layer within said circular pattern of said plurality of projections and adjacent to said point from which said injection needle extends.

13. An injection device as described in claim 12, wherein said at least one projection is configured to be displaced concurrently with a point on said conjunctival layer in a direction away from the incision created by the cutting member to form said access window free of said conjunctival layer.

14. An injection device as described in claim 10, further comprising:
a cam engaged with said injection needle hub and injection needle, wherein said cam is configured to contact said at least one projection configured to be displaced concurrently with a point on said conjunctival layer to displace said projection.

15. An injection device as described in claim 14, wherein said dilator housing further comprises:
at least one stroke window; and
said cam comprises at least one cam stop, wherein said cam stop is slidably disposed within said at least one stroke window and which limits a slidable travel of said cam, injection needle hub and injection needle.

16. An injection device as described in claim 10, further comprising:
a spring disposed between said dilator housing and said injection needle hub and injection needle.

17. An injection device as described in claim 10, wherein said at least one projection configured to be displaced concurrently with a point on said conjunctival layer is further configured to close said access window upon removal of said injection device from said conjunctival layer.

18. A method of providing an access window free of a conjunctival layer and providing an intravitreal injection through said access window, comprising:
placing a dilator assembly against a targeted intravitreal injection site and applying a pressure to said site;
applying a further pressure to said site to force a cutting member against a conjunctival layer site and cutting the conjunctival layer upon contact;
applying a further pressure to said site to force a cantilevered projection away from said incision concurrently with a point of said conjunctival layer, thereby creating an access window free of said conjunctival layer; and
advancing an injection needle into the intravitreal injection position and activating a plunger to compete an intravitreal injection through said access window.

19. A method of providing an access window free of a conjunctival layer and providing an intravitreal injection through said access window as described in claim 18, further comprising closing said access window upon removal of said dilator assembly from said conjunctival layer.

20. A method of providing an access window free of a conjunctival layer and providing a targeted intravitreal injection through said access window, comprising:
placing a first projection against a limbus site and applying a pressure to said site;
applying a further pressure to said site to force a cutting member against a conjunctival layer site and cutting the conjunctival layer upon contact;
applying a further pressure to said site to force a second projection away from said incision concurrently with a point of said conjunctival layer, thereby creating an access window free of said conjunctival layer; and
advancing an injection needle into said access window to complete an intravitreal injection through said access window,
wherein said first projection and cutting member are spaced such that said intravitreal injection occurs at a targeted point posterior to said limbus site.
